(19) United States
(12) Patent Application Publication
(10) Pub. No.: US 2009/0258843 A1
(43) Pub. Date: Oct. 15, 2009

(54) Compositions Containing Antiviral Compounds and Methods of Using the Same

(75) Inventors: Gary L. Cantrell, Troy, IL (US); Robert E. Halvachs, Belleville, IL (US); Narayanasamy Gurusamy, Ballwin, MO (US); Dawn M. Kyle, High Ridge, MO (US); Frank W. Moser, Arnold, MO (US)

Correspondence Address: Mallinckrodt Inc., 675 McDonnell Boulevard, HAZELWOOD, MO, 63042, US

(73) Assignee: Mallinckrodt Inc., Hazelwood, MO (US)

(21) Appl. No.: 12/420,261

(22) Filed: Apr. 8, 2009

Related U.S. Application Data
(60) Provisional application No. 61/044,918, filed on Apr. 15, 2008.

(51) Int. Cl. A61K 31/522; A61K 31/60; A61P 31/12

(52) U.S. Cl. 514/161; 514/263.38

(57) ABSTRACT

The present invention is directed to compositions and methods for the treatment of various diseases, pathological disorders, and medical conditions such as viral infections and cancer. The compositions include (A) an antiviral compound or a pharmaceutically acceptable salt thereof; and (B) an agent selected from the group consisting of a substituted or unsubstituted imidazole or a pharmaceutically acceptable salt thereof; a non-steroidal anti-inflammatory agent or a pharmaceutically acceptable salt thereof; an amino acid or a pharmaceutically acceptable salt thereof; a carboxylic acid or a pharmaceutically acceptable salt thereof; a sulfonic acid or a pharmaceutically acceptable salt thereof; and a combination thereof.

### FIELD OF THE INVENTION

The present invention generally relates to compositions and methods for their use in medical therapy. More particularly, the present invention relates to compositions including an antiviral compound and an agent for enhancing the solubility of the antiviral compound. The present invention also relates to methods for use of the compositions described herein for the treatment and prophylaxis of diseases, pathological disorders, and medical conditions such as, for example, viral infections and cancer.

### BACKGROUND OF THE INVENTION

Transmucosal and/or transdermal drug delivery is a relatively effective and efficient means of drug administration. Among the various advantages of such methods of drug delivery are the general avoidance of variations in the absorption and metabolism associated with oral administration and the general avoidance of the risks, inconvenience, and discomfort associated with injection. In addition, transmucosal and/or transdermal drug delivery strategies permit relatively continuous zero-order drug administration and the use of drugs with relatively short biological half-lives. These strategies can also increase the bioavailability and efficacy of drugs, as hepatic first-pass elimination is typically bypassed. Furthermore, transmucosal and transdermal treatment modalities provide a relatively simple therapeutic regime, leading to good patient compliance, which can be easily initiated and/or terminated by simple application and removal of the pharmaceutically active dose form (e.g., the patch, cream, emulsion, etc.).

Several chemical substances have been shown to possess the ability to enhance permeation across the skin, and are therefore commonly included in transmucosal and transdermal delivery systems. These include, for instance, low molecular weight alcohols (e.g., methanol, ethanol, and the like), alkyl methyl sulfoxides (e.g., decylmethylsulfoxide (DecMSO)), non-ionic surfactants (e.g., polysorbates, polyethoxylated alkyl ethers and esters and poloxamers), oleic acid, propylene glycol, Azone® (also known as 1-dodecylazacycloheptan-2-one or laurocapram), and combinations thereof. See, e.g., Rosado et al., BMC Dermatology (2003) 3:5.

Pharmaceutically active heterocycles containing substituted pyrimidine and/or purine ring systems, for example, often exhibit relatively poor solubility in conventional solvents such as water, methanol, ethanol, and aprotic dipolar solvents such as dimethylsulfoxide, dimethylacetamide, dimethylformamide, 1-methyl-2-pyrrolidinone, and the like. This physical characteristic can limit processing and pharmaceutical dose options in compositions exhibiting, for instance, effective in vitro antiviral or antitumor activity. The corresponding in vivo activity may be relatively low or ineffective due to the limited concentration of the compound delivered to the site of the disease.

The clinical benefit of antiviral compounds such as pyrimidine and/or purine-based compounds that can help mitigate acute outbreaks of DNA viruses such as Herpes Simplex Virus 1 (HSV-1), Herpes Simplex Virus 2 (HSV-2), Varicella Zoster Virus (VZV) (i.e., shingles), and cytomegalovirus is well established. As a result of its relatively poor solubility and permeation through the skin, however, the topically applied purine-based compound acyclovir is typically applied up to six times per day, or more. Due, in part, to the relatively poor bioavailability of acyclovir, derivatives of acyclovir such as valacyclovir, the L-valine ester of acyclovir, have been developed.

Various approaches have been explored in an attempt to increase the in vivo effective concentration of antiviral and antitumor compounds, particularly in topical dosage forms. Among the various permeation enhancers employed to improve the percutaneous absorption of such compounds are 50% (v/v) ethanol/water, Azone® (also known as 1-dodecylazacycloheptan-2-one or laurocapram), 95% dimethylsulfoxide with or without 5% Azone®, menthol (e.g., 1-menthol), decylmethylsulfoxide (DecMSO), histamine and histamine agonists, and capsaicin and synthetic analogs thereof (e.g., nonivamide). See, e.g., Lawanprasert et al., Mahidol Univ. J. Pharm. Sci. (2003) 30(3), 17-24; Afouna et al., Intl. J. Pharmaceutics (2003) 253, 159-68; EP 0 416 739 to Griffiths et al.; and U.S. Pub. No. 2003/0143195 to Pinsker.

Helman et al. (U.S. Pat. No. 5,164,406) (hereby incorporated by reference herein) disclose the use of certain imidazoles as dermal penetration enhancement agents in pharmaceutical formulations. Absent from the disclosure of Helman et al., however, is the use of substituted and unsubstituted imidazoles in connection with antiviral and/or antitumor agents in general, and pyrimidine- and purine-based antiviral and antitumor agents, in particular.

Although several dermal and/or mucosal penetration enhancers are known for antiviral and antitumor agents, there are limitations to their effectiveness including, for example, relatively poor solubility and/or decreased bioavailability of the pharmaceutical active. Accordingly, a need remains for improved dosage forms of antiviral and antitumor agents that promote dissolution and absorption and/or facilitate the dermal, mucosal, or other transport of the same.

### SUMMARY OF THE INVENTION

Among the various aspects of the present invention is the provision of a composition including an antiviral compound or a pharmaceutically acceptable salt thereof, in combination with one or more additional agents. In various embodiments, the antiviral compound is a pyrimidine derivative or a pharmaceutically acceptable salt thereof, a purine derivative or a pharmaceutically acceptable salt thereof, or a combination thereof. The additional agent(s) may be selected from the group consisting of a substituted or unsubstituted imidazole or a pharmaceutically acceptable salt thereof; a non-steroidal anti-inflammatory agent or a pharmaceutically acceptable salt thereof; an amino acid or a pharmaceutically acceptable salt thereof, a carboxylic acid or a pharmaceutically acceptable salt thereof, a sulfonic acid or a pharmaceutically acceptable salt thereof; and a combination thereof. The presence of the agent(s) in the composition can improve the solubility of the antiviral compound, and can facilitate the transport of the composition across biological membranes, for example, in the transdermal or transmucosal administration of the composition. In certain embodiments, the solubility-enhancing agent may have the ability to exert its own pharmacological effect (e.g., as an anti-inflammatory agent or pain reliever). Among certain other aspects of the present invention are methods for the treatment or prophylaxis of certain diseases, pathological disorders, and medical conditions such as, for instance, viral infections and cancer, using the compositions described herein.

Briefly, therefore, the present invention is directed to a composition comprising: (A) an antiviral compound or a pharmaceutically acceptable salt thereof, and (B) an agent selected from the group consisting of a substituted or unsubstituted imidazole or a pharmaceutically acceptable salt thereof; a non-steroidal anti-inflammatory agent or a pharmaceutically acceptable salt thereof; an amino acid or a pharmaceutically acceptable salt thereof, a carboxylic acid or a pharmaceutically acceptable salt thereof; a sulfonic acid or a pharmaceutically acceptable salt thereof; and a combination thereof.

The present invention is also directed to a composition comprising: (A) an antiviral compound or a pharmaceutically acceptable salt thereof; and (B) a substituted or unsubstituted imidazole or a pharmaceutically acceptable salt thereof

The present invention is also directed to a composition comprising: (A) an antiviral compound or a pharmaceutically acceptable salt thereof; (B) a substituted or unsubstituted imidazole or a pharmaceutically acceptable salt thereof; and (C) a non-steroidal anti-inflammatory agent or a pharmaceutically acceptable salt thereof.

The present invention is also directed to a composition comprising: (A) an antiviral compound or a pharmaceutically acceptable salt thereof; and (B) a non-steroidal anti-inflammatory agent or a pharmaceutically acceptable salt thereof

The present invention is also directed to a composition comprising: (A) an antiviral compound or a pharmaceutically acceptable salt thereof; and (B) an agent selected from the group consisting of a carboxylic acid or a pharmaceutically acceptable salt thereof; a sulfonic acid or a pharmaceutically acceptable salt thereof; and a combination thereof.

The present invention is also directed to a method for the prophylaxis or treatment of a viral infection, the method comprising administering to a mammal in need of such prophylaxis or treatment a prophylactic or therapeutic amount of a composition comprising: (A) an antiviral compound or a pharmaceutically acceptable salt thereof; and (B) an agent selected from the group consisting of a substituted or unsubstituted imidazole or a pharmaceutically acceptable salt thereof; a non-steroidal anti-inflammatory agent or a pharmaceutically acceptable salt thereof; an amino acid or a pharmaceutically acceptable salt thereof; a carboxylic acid or a pharmaceutically acceptable salt thereof; a sulfonic acid or a pharmaceutically acceptable salt thereof; and a combination thereof.

Other objects and features will be in part apparent and in part pointed out hereinafter.

### DETAILED DESCRIPTION OF THE INVENTION

One aspect of the present invention is directed to compositions including an antiviral compound or a pharmaceutically acceptable salt thereof and further containing one or more other agents. In various embodiments, the antiviral compound is selected from the group consisting of a pyrimidine derivative or a pharmaceutically acceptable salt thereof a purine derivative or a pharmaceutically acceptable salt thereof, and a combination thereof. The pyrimidine and/or purine derivative, for example, generally correspond to any one or more of a number of pyrimidine- or purine-based compounds having antiviral properties.

In addition to the antiviral compound(s), the compositions include at least one additional agent (i.e., a second component) that is capable of enhancing the solubility of the antiviral compound(s). In addition to the solubility-enhancing properties of the second component, such component may or may not have the ability to exert its own pharmacological effect (e.g., as a therapeutic or prophylactic agent). In one embodiment, the second component is selected from the group consisting of a substituted or unsubstituted imidazole or a pharmaceutically acceptable salt thereof; a non-steroidal anti-inflammatory agent or a pharmaceutically acceptable salt thereof; an amino acid or a pharmaceutically acceptable salt thereof; a carboxylic acid or a pharmaceutically acceptable salt thereof; a sulfonic acid or a pharmaceutically acceptable salt thereof; and a combination thereof.

The composition may include any combination of the above agent(s) in combination with the antiviral compound (e.g., the pyrimidine and/or the purine derivative). Thus, in one embodiment, for example, the composition includes: (A) an antiviral compound or a pharmaceutically acceptable salt thereof; and (B) an agent selected from the group consisting of a substituted or unsubstituted imidazole or a pharmaceutically acceptable salt thereof; a non-steroidal anti-inflammatory agent or a pharmaceutically acceptable salt thereof; an amino acid or a pharmaceutically acceptable salt thereof; a carboxylic acid or a pharmaceutically acceptable salt thereof; a sulfonic acid or a pharmaceutically acceptable salt thereof; and a combination thereof. In another embodiment, for example, the composition includes: (A) an antiviral compound or a pharmaceutically acceptable salt thereof; and (B) a substituted or unsubstituted imidazole or a pharmaceutically acceptable salt thereof. In another embodiment, the composition includes: (A) an antiviral compound or a pharmaceutically acceptable salt thereof; (B) a substituted or unsubstituted imidazole or a pharmaceutically acceptable salt thereof; and (C) a non-steroidal anti-inflammatory agent or a pharmaceutically acceptable salt thereof. In another embodiment, the composition includes: (A) an antiviral compound or a pharmaceutically acceptable salt thereof; and (B) a non-steroidal anti-inflammatory agent or a pharmaceutically acceptable salt thereof. In another embodiment, the composition includes: (A) an antiviral compound or a pharmaceutically acceptable salt thereof; and (B) an agent selected from the group consisting of an amino acid or a pharmaceutically acceptable salt thereof; a carboxylic acid or a pharmaceutically acceptable salt thereof; a sulfonic acid or a pharmaceutically acceptable salt thereof; and a combination thereof. In each of the foregoing embodiments, for example, the antiviral compound may be selected from the group consisting of a pyrimidine derivative or a pharmaceutically acceptable salt thereof, a purine derivative or a pharmaceutically acceptable salt thereof, and a combination thereof. Alternatively, in each of the foregoing embodiments the antiviral compound may be another antiviral compound such as a non-pyrimidine- or a non-purine-based compound.

It has been found that the incorporation of the second agent(s) described herein in the compositions of the present invention containing an antiviral compound (or a salt thereof) such as those having a pyrimidine and/or purine backbone can advantageously improve the solubility of the antiviral compound, and/or can facilitate the dermal, mucosal, or other transport of the antiviral compound across biological membranes, for example, in the transdermal and/or transmucosal delivery of the composition to a mammal. Additionally, or alternatively, the agent may possess one or more beneficial properties (e.g., as a therapeutic or prophylactic agent).

### Antiviral Compounds

The compositions of the present invention include an antiviral compound or a pharmaceutically acceptable salt thereof. For instance, the antiviral compound may be selected from the group consisting of a pyrimidine derivative or a pharmaceutically acceptable salt thereof, a purine derivative or a pharmaceutically acceptable salt thereof, and a combination thereof, wherein the pyrimidine- or purine-based compound(s) possess antiviral properties. Thus, where a pyrimidine derivative or a purine derivative or a combination thereof is employed, these derivatives are preferably pharmaceutically active compounds.

In general, a wide variety of antiviral compounds may be employed. The antiviral compounds that are included in the composition are generally heterocyclic compounds. The skeletal structure of the antiviral compounds typically includes a six- or nine-membered, unsaturated monocyclic or bicyclic ring substituted with one, two, three, or four nitrogen atoms. Where the antiviral compound is a pyrimidine derivative, for instance, the skeletal structure of the pyrimidine derivative typically includes a six-membered unsaturated ring with nitrogen atoms substituted at the 1- and 3-positions. Where the antiviral compound is a purine derivative, for instance, the skeletal structure of the purine derivatives typically includes a similar six-membered unsaturated ring, but the carbon atoms at the 5- and 6-positions of the six-membered ring form a fused ring system with two additional nitrogen atoms separated by one carbon atom (i.e., forming a nine-membered bicyclic ring). Other antiviral compounds may possess similar backbones as the pyrimidine or purine derivatives, but include one or more additional, or fewer, nitrogen atoms in the six- or nine-membered monocyclic or bicyclic ring. In various embodiments, the antiviral compound(s) are pharmaceutically active compounds that can be efficaciously administered to a mammal orally, topically, or by other means described in further detail below. Once administered, the pharmaceutically active compound(s) are absorbed through various biological membranes (e.g., skin, nails, mucous, or other membranes such as the gastrointestinal lining) depending on the route of administration.

The antiviral compounds which are included in the compositions of the present invention generally correspond to Formulae (1) and (2), respectively:

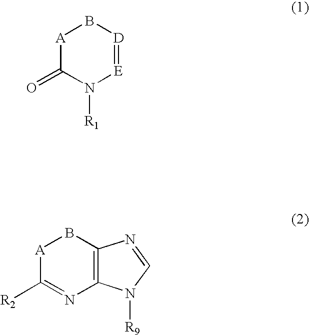

wherein:

-A-B— correspond to:

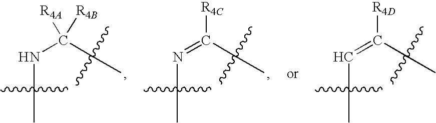

-D=E- correspond to:

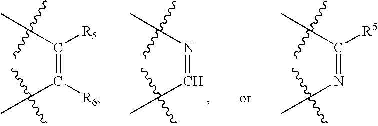

R₁ is hydrogen, heterocyclo, hydrocarbyl, or substituted hydrocarbyl;

R₂ is hydrogen, hydroxy, amino, or halo;

R_{4A} and R_{4B} are hydrogen or together form keto;

R_{4C} is hydrogen, hydroxy, amino, or alkoxy;

R_{4D} is hydroxy or alkoxy;

R₅ is hydrogen, hydrocarbyl, substituted hydrocarbyl, alkoxy, or halo;

R₆ is hydrogen or amino; and

R₉ is hydrocarbyl, substituted hydrocarbyl, or heterocyclo.

Where the antiviral compound corresponds to Formula (1), for example, in certain embodiments R₁ is heterocyclo; more preferably in these embodiments, R₁ is:

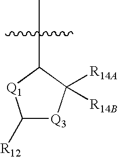

wherein:

-Q₁- is —O— or —S—;

-Q₃- is —O—, —S—, or —CH(R₁₃)—;

R₁₂ and R_{14A} are independently acyloxy, alkoxy, halo, hydrogen, hydroxy, hydrocarbyl, substituted hydrocarbyl, or N₃;

R₁₃ (if present) is acyl, acyloxy, halo, hydrogen, hydroxy, hydrocarbyl, substituted hydrocarbyl, or N₃, or together with R_{14B} forms a bond; and

R_{14B} is hydrogen or halo, or together with R₁₃ forms a bond.

In certain other embodiments in which the antiviral compound corresponds to Formula (1), R₁ is hydrocarbyl or substituted hydrocarbyl; more preferably in these embodiments, R₁ is —(CH₂)ₙ—CH(R₁₁₁)(R₁₁₂), wherein R₁₁₁ and R₁₁₂ are independently acyloxy, hydrogen, hydrocarbyl, substituted hydrocarbyl, or —O—(CH₂)ₙ—P(═O)(OH)₂, and each n is independently 1, 2, 3, or 4.

Where the antiviral compound corresponds to Formula (2), for example, in certain embodiments R₉ is hydrocarbyl or unsubstituted hydrocarbyl. Typically, such substituents contain from 1 to 20 carbon atoms and may be linear, branched or cyclic, and one or more hydrogen atoms of the substituted hydrocarbyl moieties are replaced with a different substituent (e.g., —OH, —OR, —COOH, —COOR, —CONH₂, —NH₂, —NHR, —NRR, —SH, —SR, —SO₂R, —SO₂H, —SOR, heterocyclo, and halo (including F, Cl, Br and I), among others, wherein each occurrence of R may be hydrocarbyl or substituted hydrocarbyl (e.g., substituted or unsubstituted alkyl, substituted or unsubstituted aryl, or substituted or unsubstituted aralkyl). Such substituted hydrocarbyl moieties may include substituents which correspond to particular amino acid side chain moieties including, for example, lysine, arginine, histidine, aspartic acid, glutamic acid, asparagine, glutamine, phenylalanine, tyrosine, tryptophan, cysteine, methionine, serine, and threonine, among others, as described below. In one embodiment, R₉ is substituted or unsubstituted cycloalkyl, substituted or unsubstituted cycloalkenyl, or —(CH₂)ₙ—R₉₁—R₉₂—, wherein:

R₉₁ is —(CH₂)ₙ— or —O—;

R₉₂ is —(CH₂)ₘ—CH(R₉₃)(R₉₄);

R₉₃ and R₉₄ are independently hydrogen, —(CH₂)ₙ—OR₉₅, or —(CH₂)ₙ—R₉₆;

R₉₅ is hydrogen or acyl;

R₉₆ is an amino acid ester

m is 0, 1, 2, or 3; and

each n is independently 1, 2, 3, or 4.

In certain other embodiments in which the antiviral compound corresponds to Formula (2), R₉ is heterocyclo; more preferably in these embodiments, R₉ is:

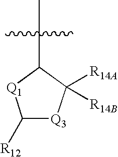

-Q₁- is —O— or —S—;

-Q₃- is —O—, —S—, or —CH(R₁₃)—

R₁₂ and R_{14A} are independently acyloxy, alkoxy, halo, hydrogen, hydroxy, hydrocarbyl, substituted hydrocarbyl, or N₃;

R₁₃ (if present) is acyl, acyloxy, halo, hydrogen, hydroxy, hydrocarbyl, substituted hydrocarbyl, or N₃, or together with R_{14B} forms a bond; and

R_{14B} is hydrogen or halo, or together with R₁₃ forms a bond.

In these and other embodiments in which the antiviral compound corresponds to Formulae (1) or (2), although R₅ may be selected from hydrogen, hydrocarbyl, substituted hydrocarbyl, alkoxy, or halo, in certain embodiments R₅ is hydrogen, substituted or unsubstituted alkyl, alkenyl, or alkynyl, or halo.

### Pyrimidines

As noted above, in certain embodiments the antiviral compound corresponds to a pyrimidine derivative. According to certain of these embodiments, for example, the pyrimidine derivatives correspond to Formula (1A):

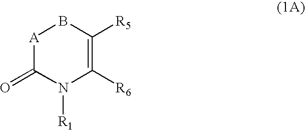

wherein:

-A-B— is:

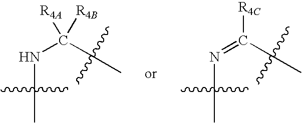

R₁ is hydrogen, heterocyclo, hydrocarbyl, or substituted hydrocarbyl;

R_{4A} and R_{4B} are hydrogen or together form keto;

R_{4C} is hydrogen, hydroxy, amino, or alkoxy;

R₅ is hydrogen, hydrocarbyl, substituted hydrocarbyl, alkoxy, or halo; and

R₆ is hydrogen or amino.

Although R₁ may be selected from hydrogen, heterocyclo, hydrocarbyl, or substituted hydrocarbyl, in certain embodiments R₁ is heterocyclo. According to these embodiments, for example, R₁ may correspond to the moiety:

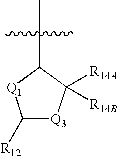

wherein:

-Q₁- is —O— or —S—;

-Q₃- is —O—, —S—, or —CH(R₁₃)—;

R₁₂ and R_{14A} are independently acyloxy, alkoxy, halo, hydrogen, hydroxy, hydrocarbyl, substituted hydrocarbyl, or N₃;

R₁₃ (if present) is acyl, acyloxy, halo, hydrogen, hydroxy, hydrocarbyl, substituted hydrocarbyl, or N₃, or together with R_{14B} forms a bond; and
R_{14B} is hydrogen or halo, or together with R₁₃ forms a bond.

Thus, for example, -Q₁- may be —O— or —S—, -Q₃- may be —S—, —O—, or —CH(R₁₃)—, R₁₂ and R_{14A} may be independently hydrogen, hydroxy, substituted or unsubstituted alkyl, aralkyl, aryl, or N₃, R₁₃ (if present) may be hydrogen, hydroxy, substituted or unsubstituted alkyl, aralkyl, aryl, or N₃, or R₁₃ together with R_{14B} forms a bond, and R_{14B} may be hydrogen, halo, or together with R₁₃ forms a bond. In particular embodiments, R₁₂ and R_{14A} are independently hydrogen, hydroxy, or substituted or unsubstituted alkyl, R₁₃ (if present) is hydroxy, halo, or N₃, or together with R_{14B} forms a bond, and R₁4B is hydrogen or halo, or together with R₁₃ forms a bond; in this embodiment R₁₂ and R_{14A} may be, for example, independently hydrogen, hydroxy, methyl, ethyl, propyl, butyl, pentyl, hexyl, or —(CH₂)ₙ—OH, wherein each n is independently 1, 2, 3, or 4.

In combination, -Q₁- may be —O— and -Q₃- may be —S—; -Q₁- may be —S— and -Q₃- may be —O—; -Q₁- may be —O— and -Q₃- may be —CH(R₁₃)-; -Q₁- may be —S— and -Q₃- may be —CH(R₁₃)—; -Q₁- and -Q₃- may both be —O—; or -Q₁- and -Q₃- may both be —S—.

As further noted in connection with Formula (1A), in certain other embodiments R₁ is hydrocarbyl or substituted hydrocarbyl; more preferably in these embodiments, R₁ is —(CH₂)ₙ—CH(R₁₁₁)(R₁₁₂), wherein R₁₁₁ and R₁₁₂ are independently acyloxy, hydrogen, hydrocarbyl, substituted hydrocarbyl, or —O—(CH₂)ₙ—P(═O)(OH)₂, and each n is independently 1, 2, 3, or 4. For example, R₁₁₁ and R₁₁₂ may be independently substituted or unsubstituted alkyl, acyloxy, aralkyl, aryl, or —O—(CH₂)—P(═O)(OH)₂. In particular embodiments, R₁₁₁ and R₁₁₂ are independently substituted or unsubstituted alkyl, acetyl, phenyl, or —O—(CH₂)ₙ—P(═O)(OH)₂; in this embodiment, for example, one of R₁₁₁and R₁₁₂ may be —(CH₂)ₙ—OH and the other of R₁₁₁ and R₁₁₂ may be —O—(CH₂)ₙ—P(═O)(OH)₂; wherein each n is independently 1, 2, 3, or 4.

As further noted in connection with Formula (1A), although R₅ may be selected from hydrogen, hydrocarbyl, substituted hydrocarbyl, alkoxy, or halo, in certain embodiments R₅ is hydrogen, substituted or unsubstituted alkyl, alkenyl, or alkynyl, or halo. For example, R₅ may be hydrogen, halo, substituted or unsubstituted methyl, ethyl, propyl, butyl, pentyl, hexyl, ethenyl, propenyl, butenyl, pentenyl, or hexenyl. In a particular embodiment, R₅ is hydrogen, halo, methyl, —C(R₅₅)₃, or —CH═CH—R₅₅, wherein R₅₅ is halo. In the embodiments in which R₅ is halo or where R₅₅ is present, the halo moiety (i.e., R₅ and/or R₅₅) may be chloro, bromo, fluoro, or iodo, typically bromo, fluoro, or iodo.

Additionally in connection with Formula (1A), R₆ is hydrogen or amino; thus, for example, R₆ could be hydrogen or —NH₂.

In addition to R₁, R₅, and R₆ as described above, the pyrimidine derivative corresponding to Formula (1A) carries the -A-B— moiety, which corresponds to:

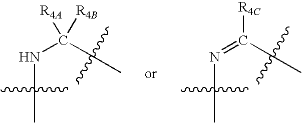

In the embodiments in which the -A-B— moiety is the former of these two structures, R_{4A} and R_{4B} are hydrogen or together form keto (i.e., ═O). In the embodiments in which the -A-B— moiety is the latter of these two structures, R_{4C} is hydrogen, hydroxy, amino, or alkoxy. Thus, in these embodiments, for example, -A-B— may correspond to:

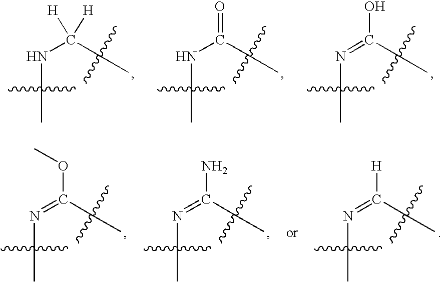

In combination, among certain of the preferred embodiments are pyrimidine derivatives corresponding to Formula (1A) wherein:

-A-B— is:

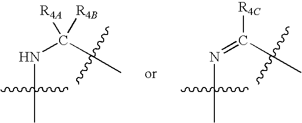

R₁ is hydrogen, heterocyclo, or —(CH₂)ₙ—CH(R₁₁₁)(R₁₁₂);

R_{4A} and R_{4B} are hydrogen or together form keto;

R_{4C} is hydrogen, hydroxy, amino, or alkoxy;

R₅ is hydrogen, hydrocarbyl, substituted hydrocarbyl, alkoxy, or halo;

R₁₁₁ and R₁₁₂ are independently hydrogen, —(CH₂)ₙ—OH, or —(CH₂)ₙ—P(═O)(OH)₂; and

each n is independently 1, 2, 3, or 4.

In certain of these preferred embodiments, R₁ is:

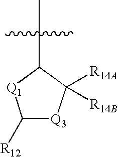

wherein -Q₁- is —O— or —S—, -Q₃- is —S—, —O—, or —CH(R₁₃)—, R₁₂ and R_{14A} are independently hydrogen, hydroxy, substituted or unsubstituted alkyl, aralkyl, aryl, or N₃, R₁₃ (if present) is hydrogen, hydroxy, substituted or unsubstituted alkyl, aralkyl, aryl, or N₃, or R₁₃ together with R_{14B} forms a bond, and R_{14B} is hydrogen, halo, or together with R₁₃ forms a bond. In each of these embodiments, for example, R₁₂ may be hydrogen, hydroxy, or —(CH₂)ₙ—OH; R₁₃ (if present) may be hydroxy, halo, or N₃, or together with R_{14B} forms a bond; R_{14A} may be hydrogen or hydroxy; and R_{14B} may be hydrogen, halo, or together with R₁₃ forms a bond; in this embodiment R₁₂ and R_{14A} may be, for example, independently hydrogen, hydroxy, methyl, ethyl, propyl, butyl, pentyl, hexyl, or —(CH₂)ₙ—OH, wherein each n is 1, 2, 3, or 4.

In combination, -Q₁- may be —O— and -Q₃- may be —S—; -Q₁- may be —S— and -Q₃- may be —O—; -Q₁- may be —O— and -Q₃- may be —CH(R₁₃)—; -Q₁- may be —S— and -Q₃- may be —CH(R₁₃)—; -Q₁- and -Q₃- may both be —O—; or -Q₁- and -Q₃- may both be —S—.

In certain embodiments, the pyrimidine derivative corresponds to Formula (1A) wherein:

-A-B— is:

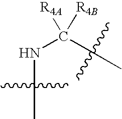

R₁ is hydrogen or:

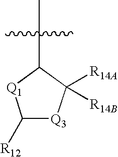

-Q₁- is —O—;

-Q₃- is —CH(R₁₃)—;

R_{4A} and R_{4B} together form keto;

R₅ is halo or —C(R₅₅)₃;

R₆ is hydrogen;

R₁₂ is hydrogen, hydroxy, alkoxy, or —(CH₂)ₙ—OH;

R₁₃ is hydroxy;

R_{14A} is hydrogen, hydroxy, or —(CH₂)ₙ—OH;

R_{14B} is hydrogen;

R₅₅ is halo; and

each n is independently 1, 2, 3, or 4. In particular, the pyrimidine derivative may be selected from the group consisting of fluorouracil, 5-deoxy-5-fluorouridine, 2-deoxy-5-fluorouridine, idoxuridine, broxuridine, trifluridine, and any combination thereof.

In other embodiments, the pyrimidine derivative corresponds to Formula (1A) wherein:

-A-B— is:

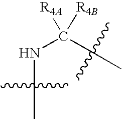

R₁ is hydrogen or:

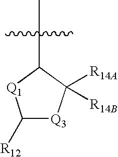

-Q₁- is —O—;

-Q₃- is —CH(R₁₃)—;

R_{4A} and R_{4B} together form keto;

R₅ is methyl or —CH═CH—R₅₅;

R₆ is hydrogen;

R₁₂ is —(CH₂)ₙ—OH;

R₁₃ is halo, hydroxy, N₃, or together with R_{14B} forms a bond;

R_{14A} is hydrogen or hydroxy;

R_{14B} is hydrogen or together with R₁₃ forms a bond;

R₅₅ is halo; and

n is independently 1, 2, 3, or 4. In particular, the pyrimidine derivative may be selected from the group consisting of sanilvudine, alovudine, sorivudine, brivudine, zidovudine, and any combination thereof

In other embodiments, the pyrimidine derivative corresponds to Formula (1A) wherein:

-A-B— is:

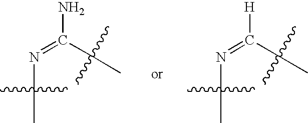

R₁ is hydrogen or —(CH₂)ₙ—CH(R₁₁₁)(R₁₁₂);

R₅ is halo or hydrogen;

R₆ is hydrogen or —NH₂;

R₁₁₁ is —(CH₂)ₙ—OH;

R₁₁₂ is —(CH₂)ₙ—P(═O)(OH)₂; and

each n is independently 1, 2, 3, or 4. In particular, the pyrimidine derivative may be selected from the group consisting of cidofovir, 5-fluorocytosine, and any combination thereof.

In other embodiments, the pyrimidine derivative corresponds to Formula (1A) wherein:

-A-B— is:

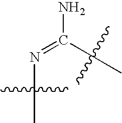

R₁ is:

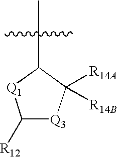

-Q₁- is —O—;

-Q₃- is —CH(R₁₃)—;

R₅ is hydrogen or halo;

R₆ is hydrogen;

R₁₂ is —(CH₂)ₙ—OH;

R₁₃ is hydrogen or hydroxy;

R_{14A} and R_{14B} are each hydrogen, R_{14A} and R_{14B} are each halo, or R_{14A} is hydroxy and R_{14B} is hydrogen; and

n is independently 1, 2, 3, or 4. In particular, the pyrimidine derivative may be selected from the group consisting of gamcitabine, arabinosylcytosine, dexelvucitabine, zalcitabine, and any combination thereof.

In other embodiments, the pyrimidine derivative corresponds to Formula (1A) wherein:

-A-B— is:

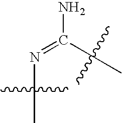

R₁ is:

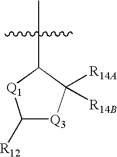

-Q₁- and -Q₃- are each —O—;

R₅ is hydrogen or halo;

R₆ is hydrogen;

R₁₂ is —(CH₂)ₙ—OH;

R_{14A} and R_{14B} are each hydrogen; and

n is independently 1, 2, 3, or 4. In particular, the pyrimidine derivative may be selected from the group consisting of troxacitabine, D-FDOC, and any combination thereof. In other embodiments, the pyrimidine derivative corresponds to Formula (1A) wherein:

-A-B— is.

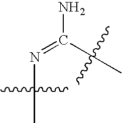

R₁ is:

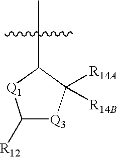

-Q₁- is —O— and -Q₃- is —S—, or -Q₁- is —S— and -Q₃- is —O—;

R₅ is hydrogen or halo;

R₆ is hydrogen;

R₁₂ is —(CH₂)ₙ—OH;

R_{14A} and R_{14B} are each hydrogen; and

n is independently 1, 2, 3, or 4. In particular, the pyrimidine derivative may be selected from the group consisting of lamivudine, racivir, apricitabine, and any combination thereof.

Representative pyrimidine derivatives falling within the structure of Formula (1A) and the various definitions for -A-B—, R₁, R₅, and R₆ described above include, for example, alovudine(3′-deoxy-3′-fluoro-thymidine), apricitabine(2(1H)-Pyrimidinone, 4-amino-1-[(2R,4R)-2-(hydroxymethyl)-1,3-oxathiolan-4-yl]-), arabinosylcytosine(2(1H)-Pyrimidinone, 4-amino-1-beta-D-arabinofuranosyl-), brivudine(5-[2-bromoethenyl]-1-[4-hydroxy-5-(hydroxymethyl)oxolan-2-yl]pyrimidine-2,4-dione), broxuridine(5-bromo-2′-deoxy-uridine), cidofovir(1-(4-amino-2-oxo-pyrimidin-1-yl)-3-hydroxy-propan-2-yl]oxymethylphosphonic acid), dexelvucitiabine(2′,3′-didehydro-2′,3′-dideoxy-5-fluoro-cytidine), D-FDOC(2(1H)-Pyrimidinone, 4-amino-5-fluoro-1-[(2R,4R)-2-(hydroxymethyl)-1,3-dioxolan-4- yl]-), fluorouracil(5-fluoro-1H-pyrimidine-2,4-dione), 2-deoxy-5-fluorouridine(5-fluoro-1-[4-hydroxy-5-(hydroxymethyl)oxolan-2-yl]pyrimidine-2,4-dione), 5-deoxy-5-fluorouridine (1-[3,4-dihydroxy-5-methyl-oxolan-2-yl]-5-fluoro-pyrimidine-2,4-dione), 5-fluorocytosine(2(1H)-6-amino-5-fluoro-pyrimidinone), gamcitabine(2′-deoxy-2′, 2′-difluoro-cytidine), idoxuridine(1-[4-hydroxy-5-(hydroxymethyl)oxolan-2-yl]-5-iodo-pyrimidine-2,4-dione), lamivudine(2,3-dideoxy-3-thiacytidine), racivir/emtricitabine (2(1H)-Pyrimidinone, 4-amino-5-fluoro-1-[(2R5S)-2-(hydroxymethyl)-1,3-oxathiolan-5-yl]-), sanilvudine(2′,3′-didehydro-3′-deoxy-thymidine), sorivudine(5-(2-bromovinyl)-1-[3,4-dihydroxy-5-(hydroxymethyl)tetrahydrofuran-2-yl]-1H-pyrimidine-2,4-dione), trifluridine(1-[4-hydroxy-5-(hydroxymethyl)oxolan-2-yl]-5-(trifluoromethyl)pyrimidine-2,4-dione), troxacitabine(2(1H)-Pyrimidinone, 4-amino-1-[(2S,4S)-2-(hydroxymethyl)-1,3-dioxolan-4-yl]-), zalcitabine(4-amino-1-[5-(hydroxymethyl)tetrahydrofuran-2-yl]-1H-pyrimidin-2-one), zidovudine (also known as azidothymidine (AZT)) (1-[4-azido-5-(hydroxymethyl) oxolan-2-yl]-5-methyl-pyrimidine-2,4-dione), pharmaceutically acceptable salts thereof, combinations thereof, and the like. In one preferred embodiment, the composition includes zidovudine or a pharmaceutically acceptable salt thereof

A pharmaceutically acceptable salt of any one or more of the pyrimidine derivatives corresponding to Formula (1A) and the various definitions for -A-B—, R₁, R₅, and R₆ described above, or a pharmaceutically acceptable salt of any of the particular pyrimidine-based antiviral agents described in the preceding paragraph, may also be included in the compositions of the present invention. Suitable salts are described in further detail below.

### Purines

As noted above, in certain embodiments the antiviral compound corresponds to a purine derivative. According to certain of these embodiments, for example, the purine derivatives correspond to Formula (2):

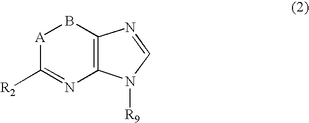

wherein:

-A-B— is:

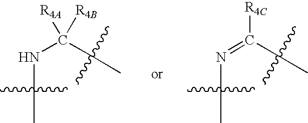

R₂ is hydrogen, amino, or halo;

R_{4A} and R_{4B} are hydrogen or together form keto;

R_{4C} is hydroxy, amino, or alkoxy; and

R₉ is hydrocarbyl, substituted hydrocarbyl, or heterocyclo.

Although R₂ may be selected from hydrogen, amino, or halo, in certain embodiments R₂ is hydrogen, —NH₂, chloro, or fluoro.

As further noted in connection with Formula (2), although R₉ may be selected from hydrocarbyl, substituted hydrocarbyl, or heterocyclo, in certain embodiments R₉ is hydrocarbyl or substituted hydrocarbyl. More preferably in these embodiments, R₉ is substituted cycloalkyl, substituted cycloalkenyl, —(CH₂)ₙ—CH(—(CH₂)_{m—CH}₃)(—O—(CH₂)ₙ—P(═O)(OH)₂), —(CH₂)ₙ—R₉₁—R₉₂—; or heterocyclo. Where R₉ is —(CH₂)ₙ—R₉₁—R₉₂, for example, R₉₁ is —O— or —(CH₂)ₙ— and R₉₂ is —(CH₂)ₘ—CH(R₉₃)(R₉₄), wherein R₉₃ and R₉₄ are independently hydrogen, —(CH₂)ₙ—OR₉₅, or —(CH₂)ₙ—R₉₆, m is 0, 1, 2, or 3, and each n is independently 1, 2, 3, or 4. Preferably, R₉₃ and R₉₄ are not both hydrogen. By way of example, one of R₉₃ and R₉₄ may be hydrogen and the other of R₉₃ and R₉₄ may be —(CH₂)ₙ—OR₉₅ or —(CH₂)ₙ—R₉₆, wherein each n is independently 1, 2, 3, or 4. Alternatively, one of R₉₃ and R₉₄ may be —(CH₂)ₙ—OR₉₅ and the other of R₉₃ and R₉₄ may be —(CH₂)ₙ—R₉₆, wherein —(CH₂)ₙ—OR₉₅, or R₉₃ and R₉₄ may both be —(CH₂)ₙ—R₉₆, wherein each n is independently 1, 2, 3, or 4.

In the embodiments in which at least one of R₉₃ and R94 is —(CH₂)ₙ—OR₉₅, R₉₅ is hydrogen or acyl. Thus, for example, R₉₅ may be hydrogen or acetyl. In the embodiments in which at least one of R₉₃ and R₉₄ is —(CH₂)ₙ—R₉₆, R₉₆ is an amino acid ester.

Where at least one of R₉₃ and R₉₄ is —(CH₂)ₙ—R₉₆ and R₉₆ is an amino acid ester, it is contemplated that esters of any naturally occurring or non-naturally occurring amino acids may be employed. This includes esters of proteogenic L-amino acids (i.e., from the 20 amino acids commonly incorporated into proteins), as well as esters of D-amino acids and esters of non-proteogenic amino acids (i.e., metabolites or analogues of proteogenic amino acids). Non-limiting examples of non-proteogenic amino acids include ornithine, taurine, hydroxyproline, hydroxylysine, norleucine, β-alanine, γ-aminobutyric acid, selenocysteine, phosphoserine, pyroglutamic acid, pyrolysine, and diketopiperazines (i.e., cyclic anhydrides of two amino acids). As noted above, the amino acid ester may also be selected from non-naturally occurring amino acids; that is, amino acid derivatives and analogs. Non-limiting examples of amino acid derivatives include selenomethionine, telluro-methionine, and p-aminophenylalanine, fluorinated amino acids (e.g., fluorinated tryptophan, tyrosine and phenylalanine), nitrophenylalanine, nitrobenzoxadiazolyl-L-lysine, deoxymethylarginine, and cyclohexylalanine. Amino acid analogs include chemically synthesized compounds having properties known in the art to be characteristic of amino acids, examples of which include, e.g., the tryptophan “analog” b-selenolo-[3,2-b]-pyrrolylalanine and the proline “analog” thiaproline (1,3-thiazolidine-4-carboxylic acid). Additional amino acid derivatives include amino acid salts, acylated amino acids, and alpha-keto amino acids.

In certain embodiments, R₉₆ is —O—C(═O)—C(NH₂)(R₉₇), wherein R₉₇ is an amino acid side chain moiety. Typically, R₉₇ is a side chain moiety from a naturally occurring amino acid. Thus, for example, R₉₇ may correspond to an amino acid side chain moiety illustrated in Table 1:

TABLE 1

| Amino Acid Side | |
|---|---|
| Chain Moiety (R₉₇) | Amino Acid |
| —H | Glycine |
| —CH₃ | Alanine |
| —CH(CH₃)₂ | Valine |
| —CH₂CH(CH₃)₂ | Leucine |
| —CH(CH₃)CH₂CH₃ | Isoleucine |
| —(CH₂)₄NH₂ | Lysine |
| —(CH₂)₃NHC(═NH)NH₂ | Arginine |
| 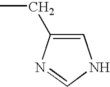 | Histidine |
| —CH₂COOH | Aspartic Acid |
| —CH₂CH₂COOHH | Glutamic Acid |
| —CH₂CONH₂ | Asparagine |
| —CH₂CH₂CONH₂ | Glutamine |
| 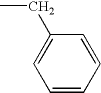 | Phenylalanine |
| 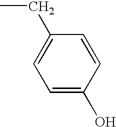 | Tyrosine |
| 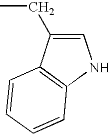 | Tryptophan |
| —CH₂SH | Cysteine |
| —CH₂CH₂SCH₃ | Methionine |
| —CH₂OH | Serine |
| —CH(OH)CH₃ | Threonine |

In a particular embodiment, R₉₇ is —CH(CH₃)₂ (i.e., R₉₆ is a valine ester); more preferably in this embodiment, R₉₆ is an L-valine ester.

For convenience purposes, only the unionized form of certain of the amino acid side chain moieties has been shown in Table 1. It is contemplated, however, that the amino acid side chain moieties illustrated in Table 1 may be utilized in the anionic, or conjugate base, form, in combination with a cation, or protonated in combination with a counterion. Suitable cations include alkali metal ions, such as sodium and potassium ions, alkaline earth metal ions, such as calcium and magnesium ions, and unsubstituted and substituted quaternary ammonium ions. The basic amino group can also be protonated in combination with a counterion such as a halogen (e.g., chloride, bromide, or iodide), acetate, formate, citrate, ascorbate, sulfate, or phosphate.

As further noted in connection with Formula (2), in certain other embodiments R₉ is heterocyclo, substituted or unsubstituted cycloalkyl, or substituted or unsubstituted cycloalkenyl. According to these embodiments, for example, R₉ may be:

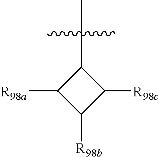

wherein R₉₈ₐ, R_{98b}, and R_{98c} are independently hydrogen, hydroxy, hydrocarbyl, or substituted hydrocarbyl. For example, R₉₈ₐ, R_{98b}, and R_{98c} may be independently hydrogen, hydroxy, or substituted alkyl. In a particular embodiment, R₉₈ₐ, R_{98b}, and R_{98c} are independently hydrogen, hydroxy, or —(CH₂)ₙ—OH, wherein each n is independently 1, 2, 3, or 4.

In certain other embodiments in which R₉ is heterocyclo, substituted or unsubstituted cycloalkyl, or substituted or unsubstituted cycloalkenyl, R₉ is:

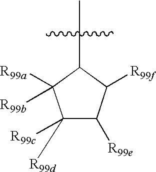

wherein R₉₉ₐ, R_{99d}, R_{99c}, and R_{99f} are independently hydrogen, hydroxy, hydrocarbyl, or substituted hydrocarbyl, and R_{99b} and R_{99c} are each hydrogen or together form a bond. More preferably in these embodiments, R₉₉ₐ, R_{99d}, R_{99c}, and R_{99f} are independently hydrogen, methylene, hydroxy, or —(CH₂)ₙ—OH, wherein each n is independently 1, 2, 3, or 4, and R_{99b} and R_{99c} are each hydrogen or together form a bond.

Where R₉ is heterocyclo, for example, in certain embodiments R₉ is:

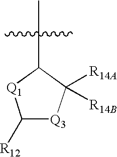

-Q₁- is —O— or —S—;

-Q₃- is —O—, —S—, or —CH(R₁₃)—;

R₁₂ and R_{14A} are independently acyloxy, alkoxy, halo, hydrogen, hydroxy, hydrocarbyl, substituted hydrocarbyl, or N₃;

R₁₃ (if present) is acyl, acyloxy, halo, hydrogen, hydroxy, hydrocarbyl, substituted hydrocarbyl, or N₃, or together with R_{14B} forms a bond; and

R_{14B} is hydrogen or halo, or together with R₁₃ forms a bond.

Thus, for example, -Q₁- may be —O— or —S—, -Q₃- may be —S—, —O—, or —CH(R₁₃)—, R₁₂ and R_{14A} may be independently hydrogen, hydroxy, substituted or unsubstituted alkyl, aralkyl, aryl, or N₃, R₁₃ (if present) may be hydrogen, hydroxy, substituted or unsubstituted alkyl, aralkyl, aryl, or N₃, or R₁₃ together with R_{14B} forms a bond, and R_{14B} may be hydrogen, halo, or together with R₁₃ forms a bond. In a particular embodiment, R₁₂ and R_{14A} are independently hydrogen, hydroxy, or substituted or unsubstituted alkyl, R₁₃ (if present) is hydroxy, halo, or N₃, or together with R_{14B} forms a bond, and R_{14B} is hydrogen or halo, or together with R₁₃ forms a bond; in this embodiment R₁₂ and R_{14A} may be, for example, independently hydrogen, hydroxy, methyl, ethyl, propyl, butyl, pentyl, hexyl, or —(CH₂)ₙ—OH, wherein each n is 1, 2, 3, or 4.

In combination, -Q₁- may be —O— and -Q₃- may be —S—; -Q₁- may be —S— and -Q₃- may be —O—; -Q₁- may be —O— and -Q₃- may be —CH(R₁₃)—; -Q₁- may be —S— and -Q₃- may be —CH(R₁₃)—; -Q₁- and -Q₃- may both be —O—; or -Q₁- and -Q₃- may both be —S—.

In addition to R₂ and R₉ described above, the purine derivative corresponding to Formula (2) carries the -A-B— moiety, which corresponds to:

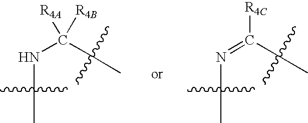

In the embodiments in which the -A-B— moiety is the former of these two structures, R_{4A} and R_{4B} are hydrogen or together form keto (i.e., ═O). In the embodiments in which the -A-B— moiety is the latter of these two structures, R_{4C} is hydroxy, amino, or alkoxy. Thus, in these embodiments, for example, -A-B— may correspond to:

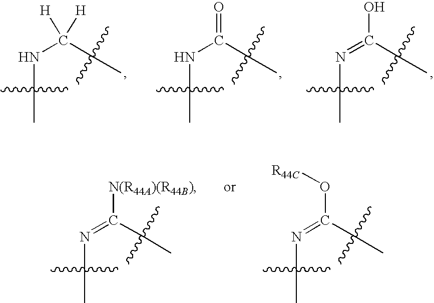

wherein R_{44A} and R_{44D} are independently hydrogen, hydrocarbyl, substituted hydrocarbyl, heteroaryl, or heterocyclo, or R_{44A} and R_{44B} taken together form a substituted or unsubstituted alicyclic, aryl, or heterocyclic moiety, and R_{44C} is alkyl (e.g., methyl). Typically, R_{44A} and R_{44B} are each hydrogen or one of R_{44A} and R_{44B} is hydrogen and the other of R_{44A} and R_{44B} is alkyl (e.g., cycloalkyl).

In combination, among certain of the preferred embodiments are purine derivatives corresponding to Formula (2) wherein:

-A-B— is:

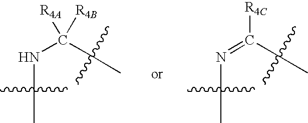

R₂ is hydrogen, —NH₂, or halo;

R_{4A} and R_{4B} are hydrogen or together form keto;

R_{4C} is hydroxy, —NH₂, or alkoxy;

R₉ is substituted cycloalkyl, substituted cycloalkenyl, —(CH₂)ₙ—CH(—(CH₂)ₘ—CH₃)(—O—(CH₂)ₙ—P(═O)(OH)₂), —(CH₂)ₙ—R₉₁—R₉₂—, or heterocyclo;

R₉₁ is —O— or —(CH₂)ₙ—;

R₉₂ is —(CH₂)ₘ—CH(R₉₃)(R₉₄);

R₉₃ and R₉₄ are independently hydrogen, —(CH₂)ₙ—OR₉₅, or —(CH₂)ₙ—R₉₆;

R₉₅ is hydrogen or acyl;

R₉₆ is an amino acid ester;

each m is independently 0, 1, 2, or 3; and

each n is independently 1, 2, 3, or 4.

In certain of these preferred embodiments, R₉ is:

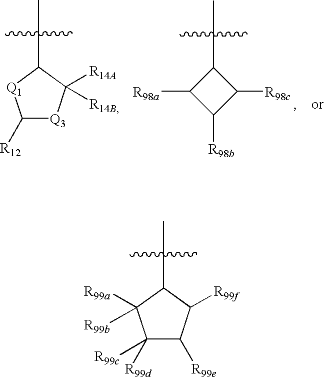

wherein:

-Q₁- is —O— or —S—;

-Q₃- is —S—, —O—, or —CH(R₁₃)—;

R₁₂ and R_{14A} are independently acyloxy, alkoxy, halo, hydrogen, hydroxy, substituted or unsubstituted alkyl, aralkyl, aryl, or N₃;

R₁₃ (if present) is acyl, acyloxy, hydrogen, hydroxy, substituted or unsubstituted alkyl, aralkyl, aryl, or N₃, or R₁₃ together with R_{14B} forms a bond;

R_{14B} is hydrogen, halo, or together with R₁₃ forms a bond;

R₉₈ₐ, R_{98b}, and R_{98c} are independently hydrogen, hydroxy, or —(CH₂)ₙ—OH;

R₉₉ₐ, R_{99d}, R₉₉ₑ, and R_{99f} are independently hydrogen, methylene, hydroxy, or —(CH₂)ₙ—OH;

R_{99b} and R_{99c} are each hydrogen or together form a bond; and

each n is independently 1, 2, 3, or 4.

In some embodiments, the purine derivative corresponds to Formula (2) wherein:

-A-B— is:

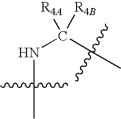

R₂ is —NH₂;

R_{4A} and R_{4B} are hydrogen or together form keto;

R₉ is —(CH₂)ₙ—R₉₁-R₉₂;

R₉₁ is —(CH₂)ₙ— or —O—;

R₉₂ is —(CH₂)ₘ—CH(R₉₃)(R₉₄);

R₉₃ and R₉₄ are independently —(CH₂)ₙ—OR₉₅ or —(CH₂)ₙ—R₉₆;

R₉₅ is hydrogen or acetyl;

R₉₆ is an amino acid ester;

m is 0, 1, 2, or 3; and

each n is independently 1, 2, 3, or 4. In particular, the purine derivative may be selected from the group consisting of ganciclovir, famciclovir, penciclovir, valganciclovir, and any combination thereof.

In other embodiments, the purine derivative corresponds to Formula (2) wherein:

-A-B— is:

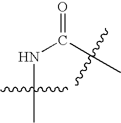

R₂ is —NH₂;

R₉ is —(CH₂)ₙ—R₉₁—R₉₂;

R₉₁ is —O—;

R₉₂ is —(CH₂)ₘ—CH(R₉₃)(R₉₄);

one of R₉₃ and R₉₄ is hydrogen and the other of R₉₃ and R₉₄ is —(CH₂)ₙ—OR₉₅ or —(CH₂)ₙ—R₉₆;

R₉₅ is hydrogen;

R₉₆ is an amino acid ester;

m is 0, 1, 2, or 3; and

each n is independently 1, 2, 3, or 4. In particular, the purine derivative may be selected from the group consisting of acyclovir, valacyclovir, desciclovir, and any combination thereof.

In other embodiments, the purine derivative corresponds to Formula (2) wherein:

-A-B— is:

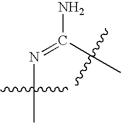

R₂ is hydrogen, -NH₂, chloro, or fluoro;

R₉ is —(CH₂)ₙ—CH(—(CH₂)ₘ—CH₃)(—O—(CH₂)ₙ—P(═O)(OH)₂) or:

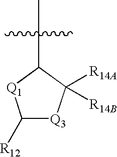

-Q₁- is —O—;

-Q₃- is —O— or —CH(R₁₃)—;

R₁₂ and R_{14A} are independently hydrogen, hydroxy, halo, alkoxy, or —(CH₂)ₙ—OH;

R₁₃ is hydroxy;

R_{14B} is hydrogen;

m is 0, 1, 2, or 3; and

each n is independently 1, 2, 3, or 4. In particular, the purine derivative may be selected from the group consisting of fludarabine, vidarabine, clofarabine, amdoxovir, tenofovir, and any combination thereof.

In other embodiments, the purine derivative corresponds to Formula (2) wherein:

-A-B— is:

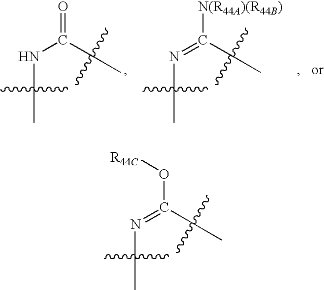

R₂ is hydrogen or —NH₂;

R₉ is:

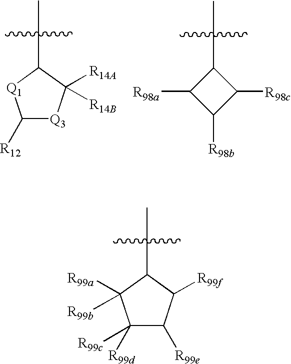

-Q₁- is —O—;

-Q₃- is —O— or —CH(R₁₃)—;

R₁₂ and R_{14A} are independently hydrogen, hydroxy, or —(CH₂)ₙ—OH;

R₁₃ is hydrogen or hydroxy;

R_{14B} is hydrogen;

R_{44A} is cycloalkyl;

R_{44B} is hydrogen;

R_{44C} is methyl;

R₉₈ₐ, R_{98b}, and R_{98c} are independently hydrogen or —(CH₂)ₙ—OH;

R₉₉ₐ is hydrogen;

R_{99b} is hydrogen or together with R_{99c} forms a bond;

R_{99c} is hydrogen or together with R_{99b} forms a bond;

R_{99d} is hydrogen or hydroxy;

R₉₉ₑ is —(CH₂)ₙ—OH;

R_{99f} is hydrogen or ═CH₂; and

each n is independently 1, 2, 3, or 4. In particular, the purine derivative may be selected from the group consisting of lobucavir, entecavir, abacavir, nelarabine, didanosine, and any combination thereof.

Representative purine derivatives falling within the structure of Formula (2) and the various definitions for -A-B—, R₂, and R₉ above include, for example, abacavir(2-Cyclopentene-1-methanol, 4-[2-amino-6-(cyclopropylamino)-9H-purin-9-yl]-, (1S,4R)-), acyclovir(2-amino-9-(2-hydroxyethoxymethyl)-3H-purin-6-one), amdoxovir(1,3-Dioxolane-2-methanol, 4-(2,6-diamino-9H-purin-9-yl)-, (2R,4R)-), clofarabine(9H-Purin-6-amine, 2-chloro-9-(2-deoxy-2-fluoro-β-D-arabinofuranosyl)-), desciclovir(2-[(2-aminopurin-9-yl)methoxy]ethanol), didanosine(9-[5-(hydroxymethyl)oxolan-2-yl]-3H-purin-6-one), famciclovir([2-(acetyloxymethyl)-4-(2-aminopurin-9-yl)-butyl]acetate), entecavir(6H-Purin-6-one, 2-amino-1,9-dihydro-9-[(1S,3R,4S)-4-hydroxy-3-(hydroxymethyl)-2-methylenecyclopentyl]-), fludarabine(9H-Purin-6-amine, 9-p-D-arabinofuranosyl-2-fluoro-), ganciclovir(2-amino-9-(1,3-dihydroxypropan-2-yloxymethyl)-3H-purin-6-one), lobucavir(2-amino-9-[2,3-bis(hydroxymethyl)cyclobutyl]-3H-purin-6-one), nelarabine(9H-Purin-2-amine, 9-β-D-arabinofuranosyl-6-methoxy-), penciclovir(2-amino-9-[4-hydroxy-3-(hydroxymethyl)butyl]-3H-purin-6-one), tenofovir(Phosphonic acid, P-[[(1R)-2-(6-amino-9H-purin-9-yl)-1-methylethoxy]methyl]-), valacyclovir(2-[(2-amino-6-oxo-3,9-dihydropurin-9-yl)methoxy]ethyl-2-amino-3-methyl-butanoate), valganciclovir(2-[(2-amino-6-oxo-3,6-dihydro-9H-purin-9-yl)methoxy]-3-hydroxypropyl-2-amino-3-methyl-butanoate), vidarabine(2-(6-aminopurin-9-yl)-5-(hydroxymethyl)oxolane-3,4-diol hydrate), pharmaceutically acceptable salts thereof combinations thereof, and the like. In one preferred embodiment, the composition includes acyclovir or a pharmaceutically acceptable salt thereof

A pharmaceutically acceptable salt of any one or more of the purine derivatives corresponding to Formula (2) and the various definitions for -A-B—, R₂, and R₉ described above, or a pharmaceutically acceptable salt of any of the particular purine-based antiviral agents described in the preceding paragraph, may also be included in the compositions of the present invention. Suitable salts are described in further detail below.

### Other Antiviral Agents

Still other antiviral agents that may be employed in the compositions of the invention include those corresponding to Formula (1) wherein:

-A-B— is:

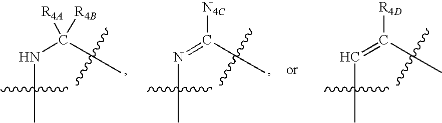

-D=E- is:

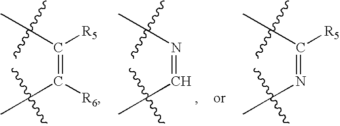

R₁ is:

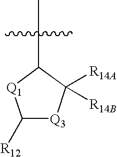

-Q₁- is —O— or —S—;

-Q₃- is —S—, —O—, or —CH(R₁₃)—;

R_{4A} and R_{4B} are hydrogen or together form keto;

R_{4C} is amino;

R_{4D} is hydroxy;

R₅ and R₆ are hydrogen;

R₁₂ and R_{14A} are independently acyloxy, alkoxy, halo, hydrogen, hydroxy, substituted or unsubstituted alkyl, aralkyl, aryl, or N₃;

R₁₃ (if present) is acyl, acyloxy, hydrogen, hydroxy, substituted or unsubstituted alkyl, aralkyl, aryl, or N₃, or R₁₃ together with R_{14B} forms a bond; and

R_{14B} is hydrogen, halo, or together with R₁₃ forms a bond.

In some embodiments, the antiviral compound corresponds to Formula (1) wherein:

-A-B— is:

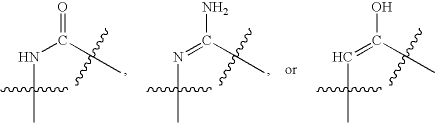

-D=E- is:

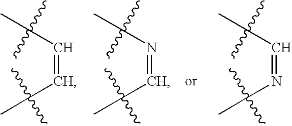

R₁ is:

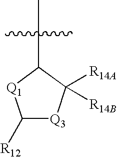

-Q₁- is —O—;

-Q₃- is —CH(R₁₃)—;

R₁₂ is —(CH₂)ₙ—OH;

R₁₃ is hydroxy;

R_{14A} is hydrogen or hydroxy;

R_{14B} is hydrogen; and

n is 1, 2, 3, or 4. In particular, the antiviral agent may be selected from the group consisting of deazauridine, riboazauracil, decitabine, and any combination thereof.

Among certain of the preferred aspects of the invention are compositions in which the antiviral agent is selected from the compounds in Table 2.

TABLE 2

| Name(s): | Structure: |
|---|---|
| CA Index Name: 2(1H)-Pyrimidinone, 4-amino-1-[(2R,4R)-2-(hydroxymethyl)-1,3- oxathiolan-4-yl]- Other Names: 2(1H)-Pyrimidinone, 4- amino-1-[2-(hydroxymethyl)-1,3-oxathiolan- 4-yl]-, (2R-cis)-; 1,3-Oxathiolane, 2(1H)- pyrimidinone deriv.; (−)-2′-Deoxy-3′-oxa-4′- thiocytidine; (−)-BCH 10652; AVX 754; Apricitabine; BCH 10618; SPD 754 | 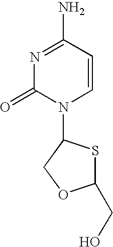 |
| CA Index Name: 2(1H)-Pyrimidinone, 4- amino-5-fluoro-1-[(2R,5S)-2- (hydroxymethyl)-1,3-oxathiolan-5-yl]-, rel- Other Names: 2(1H)-Pyrimidinone, 4- amino-5-fluoro-1-[2-(hydroxymethyl)-1,3- oxathiolan-5-yl]-, cis-(±)-; 1,3-Oxathiolane, 2(1H)-pyrimidinone deriv.; 2′,3′-Dideoxy-5- fluoro-3′-thiacytidine; 2(1H)-Pyrimidinone, 4- amino-5-fluoro-1 -[2-(hydroxymethyl)-1,3- oxathiolan-5-yl]-, cis-; 3′-Thia-2′,3′-dideoxy- 5-fluorocytidine; FTC; Racivir | 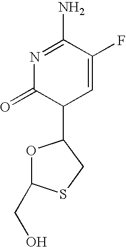 |
| CA Index Name: Thymidine, 2,3′- didehydro-3′-deoxy- Other Names: Thymine, 1-(2,3-dideoxy-β- D-glycero-pent-2-enofuranosyl)- (7CI,8CI); 2′-Thymidinene, 3′-deoxy-(8CI); 2′,3′- Didehydro-3′-deoxythymidine; 3′-Deoxy-2′,3′- didehydrothymidine; BMY 27857; D 4T; D 4T (nucleoside); NSC 163661; Sanilvudine; Stavir; Stavudine; Zerit | 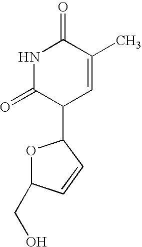 |
| CA Index Name: Cytidine, 2′,3′-dideoxy- Other Names: 2′,3′-Dideoxycytidine; D 2C; Dideoxycytidine; Hivid; NSC 606170; Ro 24-2027/000; Zalcitabine; ddC | 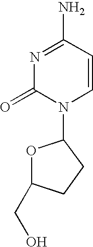 |
| CA Index Name: 2(1H)-Pyrimidinone, 4- amino-5-fluoro-1-[(2R,4R)-2- (hydroxymethyl)-1,3-dioxolan-4-yl]- Other Names: 2(1H)-Pyrimidinone, 4- amino-5-fluoro-1-[2-(hydroxymethyl)-1,3- dioxolan-4-yl]-, (2R-cis)-; β-D-Dioxolane-5- fluorocytidine; D-FDOC | 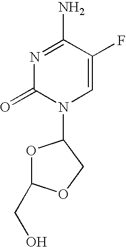 |
| CA Index Name: 2(1H)-Pyrimidinone, 4- amino-5-fluoro-1-[(2R,5S)-2- (hydroxymethyl)-1,3-oxathiolan-5-yl]- Other Names: 2(1H)-Pyrimidinone, 4- amino-5-fluoro-1-[2-(hydroxymethyl)-1,3- oxathiolan-5-yl]-, (2R-cis)-; 1,3-Oxathiolane, 2(1H)-pyrimidinone deriv.; (−)-2′,3′-Dideoxy- 5-fluoro-3′-thiacytidine; (−)-2′-Deoxy-5- fluoro-3′-thiacytidine; (−)-FTC; 524W91; BW 1592; BW 524W91; Coviracil; Emitnicibatine; Emitricitabine; Emtricitabine; Emtriva; FTC, (−)- | 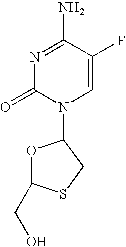 |
| CA Index Name: Cytidine, 2′,3′-didehydro- 2′,3′-dideoxy-5-fluoro- Other Names: D-d4FC; DOC 817; DPC 817; Dexelvucitabine; INCB 8721; RA 131423; Reverset; YZ 817 | 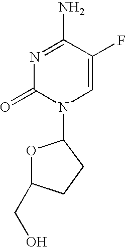 |
| CA Index Name: 2,4(1H,3H) Pyrimidinedione, 5-fluoro- Other Names: Uracil, 5-fluoro- (8CI); 2,4-Dihydroxy-5-fluoropyrimidine; 2,4 Dioxo-5-fluoropyrimidine; 5-FU; 5-Fluoracyl; 5-Fluoro-2,4(1H,3H)-pyrimidinedione; 5- Fluoro-2,4-pyrimidinedione; 5- Fluoropyrimidine-2,4-diol; 5-Fluorouracil; Adrucil; Arumel; Carac; Carzonal; Efudex; Efudix; Efurix; FU; Fluoroblastin; Fluoroplex; Fluorouracil; Flurablastin; Fluracedyl; Fluracil; Fluracilum; Fluri; Fluril; Ftoruracil; Kecimeton; NSC 19893; Phthoruracil;Phtoruracil; Queroplex, Ro 2- 9757; Timazin; U 8953; Ulup | 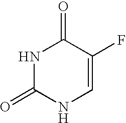 |
| CA Index Name: Uridine, 2′-deoxy-5- fluoro- Other Names: 1-(2-Deoxy-β-D- ribofuranosyl)-5-fluorouracil; 2′-Deoxy-5- fluorouridine; 5-Fluoro-2′-deoxy-β-uridine; 5- Fluoro-2′-deoxyuridine; 5-Fluorodeoxyuridine; 5-Fluorouracil 2′-deoxyriboside; 5-Fluorouracil deoxyriboside; FUDR; FdUrd; Floxuridin; Floxuridine; NSC 26740; NSC 27640 | 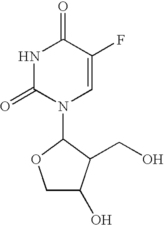 |
| CA Index Name: (2(1H)-Pyrimidinone, 4- amino-1-beta-D-arabinofuranosyl-) Other Names: arabinosylcytosine | 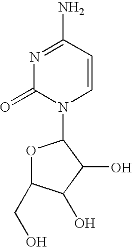 |
| CA Index Name: Cytidine, 2′-deoxy-2′,2′- difluoro- Other Names: 2′,2′-Difluoro-2′- deoxycytidine; 2,2′-Difluorodeoxycytidine; 2′-Deoxy-2′,2′-difluorocytidine; DDFC; DFdC; DFdCyd; Folfugem; Gamcitabine; Gemcitabine; LY 188011; NSC 613327 | 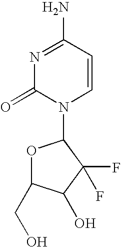 |
| CA Index Name: 2(1H)-Pyrimidinone, 4- amino-1-[(2S,4S)-2-(hydroxymethyl)-1,3- dioxolan-4-yl]- Other Names: 2(1H)-Pyrimidinone, 4- amino-1-[2-(hydroxymethyl)-1,3-dioxolan-4- yl]-, (2S-cis)-; (−)-BCH 204; (−)-OccC; α-L- Dioxolane-C; BCH 4556; L-OddC; SPD 758; Troxacitabine; Troxatyl | 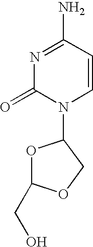 |
| CA Index Name: 1,2,4-Triazine- 3,5(2H,4H)-dione, 2-β-D-ribofuranosyl- Other Names: as-Triazine- 3,5(2H,4H)-dione, 2-β-D-ribofuranosyl- (6CI,7CI,8CI); 2-β-D-Ribofuranosyl-as- triazine-3,5(2H,4H)-dione; 6-Azauracil 1- riboside; 6-Azauracil riboside; 6-Azauracil-β- D-riboside; 6-Azauridine; 6-Azur; 6- Azuridine; AzUR; Azauridine; NSC 32074; Rib-Azauracil; Ribo-azuracil; Riboazauracil; ribo-Azauracil | 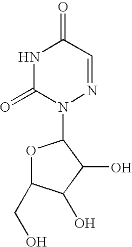 |
| CA Index Name: Uridine, 2′-deoxy-5-iodo- Other Names: 1-(2-Deoxy-β-D- ribofuranosyl)-5-iodouracil; 2′-Deoxy-5- iodouridine; 5-Iodo-2′-deoxyuridine; 5-Iodo- 2′-desoxyuridine; 5-Iododeoxyuridine; 5- Iodouracil deoxyriboside; 5IUDR; Allergan 211; Dendrid; Emanil; Herpe-Gel; Herpesil; Herpidu; Herplex; IDU; IDUR; IUDR; Idexur; Idoxene; Idoxuridin; Idoxuridine; Idu Oculos; Iducher; Idulea; Iduridin; Iododeoxyuridine; Joddeoxiuridin; Kerecid; NSC 39661; Ophthalmadine; SKF 14287; Stoxil; Synmiol; Virudox | 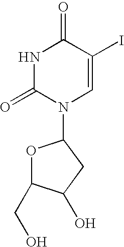 |
| CA Index Name: Uridine, 5-bromo-2′- deoxy- Other Names: 2′-Deoxy-5- bromouridine; 5-BDU; 5-Bromo-2′- deoxyuridine; 5-Bromo-2′-desoxyuridine; 5-Bromodeoxyuridine; 5-Bromodesoxyuridine; 5-Bromouracil deoxyriboside; 5-Bromouracil-2- deoxyriboside; BRUDR; BUdR; Bromodeoxyuridine; Broxuridine; NSC 38297 | 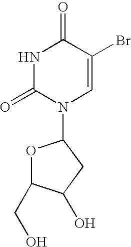 |
| CA Index Name: 2(1H)-Pyrimidinone, 6-amino-5-fluoro- Other Names: 2(1H)-Pyrimidinone, 4-amino-5-fluoro- (9CI); Cytosine, 5-fluoro- (6CI,7CI,8CI); 4-Amino-5-fluoropyrimidin- 2(1H)-one; 5-Fluorocytosin; 5-Fluorocytosine; 6-Amino-2-oxo-5- fluoropyrimidine; Alcobon; Ancobon; Ancotil; Ancotyl; Flucytosin; Flucytosine; Fluocytosine; Fluorocytosine; NSC 103805; Ro 2-9915 | 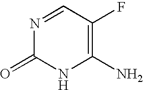 |
| CA Index Name: 2(1H)-Pyridinone, 4- hydroxy-1-β-D-ribofuranosyl- Other Names: 2(1H)-Pyridone, 4- hydroxy-1-β-D-ribofuranosyl- (8CI); 1-β-D- Ribofuranosyl-4-hydroxy-2-pyridone; 3- Deazauridine; 3-Deazuridine; 4-Hydroxy-1- (β-D-ribofuranosy1)-2-pyridone; NSC 126849 | 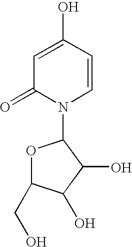 |
| CA Index Name: 1,3,5-Triazin-2(1H)-one, 4-amino-1-(2-deoxy-β-D-erythro- pentofuranosyl)- Other Names: s-Triazin-2(1H)-one, 4- amino-1-(2-deoxy-β-D-erythro- pentofuranosyl)- (7CI,8CI); 2′-Deoxy-5- azacytidine; 2-Desoxy-5-azacytidine; 5-Aza- 2-deoxycytidine; 5-Azadeoxycytidine; DAC; Dacogen; Decitabine; NSC 127716 | 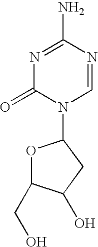 |
| CA Index Name: 1-[3,4-dihydroxy-5-methyl- oxolan-2-yl]-5-fluoro-pyrimidine-2,4-dione Other Names: 5-deoxy-5-fluorouridine | 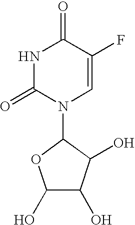 |
| CA Index Name: 1-[4-hydroxy-5- (hydroxymethyl)oxolan-2-yl]-5- (trifluoromethyl)pyrimidine-2,4-dione Other Names: trifluridine | 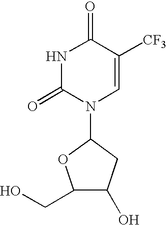 |
| CA Index Name: 5-(2-bromovinyl)-1-[3,4- dihydroxy-5-(hydroxymethyl)tetrahydrofuran- 2-yl]-1H-pyrimidine-2,4-dione Other Names: sorivudine | 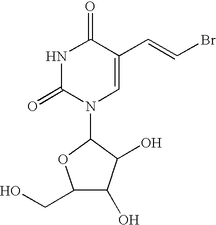 |
| CA Index Name: 5-[2-bromoethenyl]-1-[4- hydroxy-5-(hydroxymethyl)oxolan-2- yl]pyrimidine-2,4-dione Other Names: brivudine | 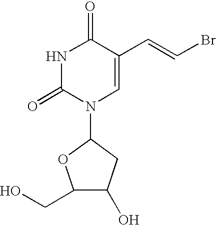 |
| CA Index Name: 1-(4-amino-2-oxo- pyrimidin-1-yl)-3-hydroxy-propan-2-yl] oxymethylphosphonic acid Other Names: cidofovir | 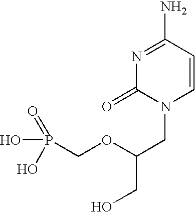 |
| CA Index Name: Thymidine, 3′-deoxy-3′- fluoro- Other Names: 1-(3′-Deoxy-3′-fluoro- β-D-pentofuranosyl)thymine; 3′-Deoxy-3′- fluorothymidine; 3′-Fluoro -3′- deoxythymidine; 3′-Fluorodeoxythymidine; 3′-Fluorothymidine; Alovudine; CL 184824; FLT; MIV 310; NSC 140025 | 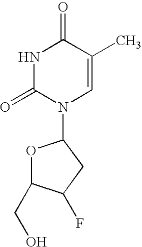 |
| CA Index Name: 2(1H)-Pyrimidinone, 4- amino-1-[(2R,5S)-2-(hydroxymethyl)-1,3- oxathiolan-5-yl]- Other Names: 2(1H)-Pyrimidinone, 4- amino-1-[2-(hydroxymethyl)-1,3-oxathiolan- 5-yl]-, (2R-cis)-; 1,3-Oxathiolane, 2(1H)- pyrimidinone deriv.; (−)-2′-Deoxy-3′- thiacytidine; (−)-BCH 189; β-L-2′,3′-Dideoxy- 3′-thiacytidine; β-L-3′-Thia-2′,3′- dideoxycytidine; 3TC; BCH 189, (−)-; Epivir; Epivir HBV; GR 109714X; Hepitec; Heptovir; L-SddC; Lamivir; Lamivudine; Zeffix; Zefix | 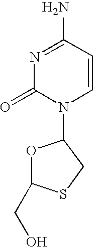 |
| CA Index Name: Thymidine, 3′-azido-3′- deoxy- Other Names: 3′-Azido-3′- deoxythymidine; 3′-Azidothymidine; 3′- Deoxy-3′-azidothymidine; 3-Azido-3- deoxythymidine; AZT; AZT (pharmaceutical); Azidothymidine; Azitidin; BW-A 509U; Compound S; NSC 602670; Retrovir; Retrovir IV; Timazid; Viro-Z; ZDV; ZVD; Zido-H; Zidovudine | 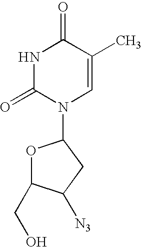 |
| CA Index Name: 2-Cyclopentene-1- methanol, 4-[2-amino-6-(cyclopropylamino)- 9H-purin-9-yl]-, (1S,4R)- Other Names: 2-Cyclopentene-1- methanol, 4-[2-amino-6-(cyclopropylamino)- 9H-purin-9-yl]-, (1S-cis)-; 1592U89; Abacavir; Ziagen | 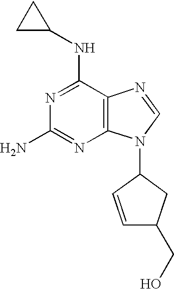 |
| CA Index Name: 6H-Purin-6-one, 2-amino- 1,9-dihydro-9-[[2-hydroxy-1- (hydroxymethyl)ethoxy]methyl]- Other Names: 2′-NDG; 2′-Nor-2′- deoxyguanosine; 9-(1,3-Dihydroxy-2- propoxymethyl)guanine; BW 759; BW 759U; BW-B 759U; Biolf 62; DHPG; Ganciclovir; Gancyclovir; HHEMG; Hydroxyacyclovir; RS 21592; Vitrasert | 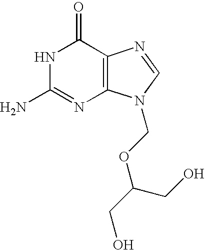 |
| CA Index Name: 1,3-Propanediol, 2-[2-(2- amino-9H-purin-9-yl)ethyl]-, 1,3-diacetate Other Names; 1,3-Propanediol, 2-[2- (2-amino-9H-purin-9-yl)ethyl]-, diacetate (ester) (9CI); 9-[4-Acetoxy-3- (acetoxymethyl)but-1-yl]-2-aminopurine; 9- [4-Acetoxy-3-(acetoxymethyl)butyl]-2- aminopurine; BRL 42810; FCV; Famciclovir; Famcivir; Fameyclovir; Famtrex; Famvir | 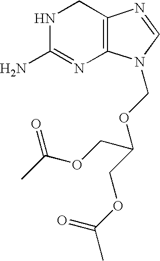 |
| CA Index Name: Inosine, 2′,3′-dideoxy- Other Names: 2′,3′-Dideoxyinosine; BMY 40900; DdI; DdI (nucleoside); Didanosine; Dideoxyinosine; NSC 612049; Videx; Videx EC | 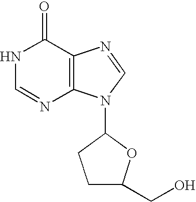 |
| CA Index Name: 6H-Purin-6-one, 2-amino- 1,9-dihydro-9-[4-hydroxy-3- (hydroxymethyl)butyl]- Other Names: 9-[4-Hydroxy-3- (hydroxymethyl)butyl]guanine; BRL 39123; Denavir; Penciclovir; VSA 671 | 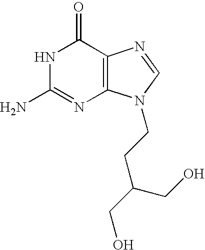 |
| CA Index Name: Phosphonic acid, P-[[(1R)- 2-(6-amino-9H-purin-9-yl)-1- methylethoxy]methyl]- Other Names: Phosphonic acid, [[(1R)-2-(6-amino-9H-purin-9-yl)-1- methylethoxylmethyl]-(9CI); Phosphonic acid, [[2-(6-amino-9H-purin-9-yl)-1- methylethoxylmethyl]-, (R)-; (R)-9-(2- Phosphonomethoxypropyl)adenine; GS 1275; GS 1278; PMPA; Tenefovir; Tenofovir | 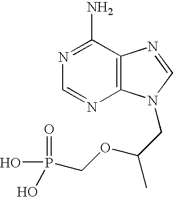 |
| CA Index Name: 1,3-Dioxolane-2-methanol, 4-(2,6-diamino-9H-purin-9-yl)-, (2R,4R)- Other Names: 1,3-Dioxolane-2- methanol, 4-(2,6-diamino-9H-purin-9-yl)-, (2R-cis)-; (−)-DAPD; β-D-2,6-Diaminopurine- dioxolane; Amdoxovir; DAPD | 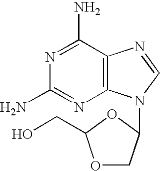 |
| CA Index Name: 6H-Purin-6-one, 2-amino- 1,9-dihydro-9-[(1S,3R,4S)-4-hydroxy-3- (hydroxymethyl)-2-methylenecyclopentyl]- Other Names: 6H-Purin-6-one, 2- amino-1,9-dihydro-9-[4-hydroxy-3- (hydroxymethyl)-2-methylenecyclopentyl]-, [1S-(1α,3α,4β]-; BMS 200475; Baraclude; Entecavir; SQ 34676 | 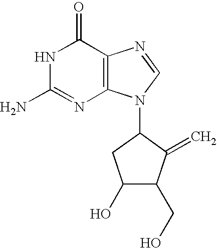 |
| CA Index Name: 9H-Purin-6-amine, 9-β-D- arabinofuranosyl-2-fluoro- Other Names: Adenine, 9-β-D- arabinofuranosyl-2-fluoro- (8CI); 2-Fluoro Ara-A; 2-Fluoro-9-β-D- arabinofuranosyladenine; 2-Fluoroadenine arabinoside; 9-β-D-Arabinofuranosyl-2- fluoroadenine; 9-β-D-Arabinosyl-2- fluoroadenine; F-ara-A; Fludarabine; NSC 118218; NSC 118218H | 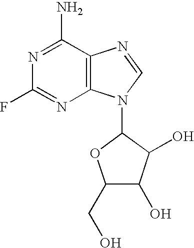 |
| CA Index Name: 9H-Purin-6-amine, 2- chloro-9-(2-deoxy-2-fluoro-β-D- arabinofuranosyl)- Other Names: 2-Chloro-9-(2-deoxy-2- fluoro-β-D-arabinofuranosyl)adenine; Clofarabine; Clofarex; Clolar | 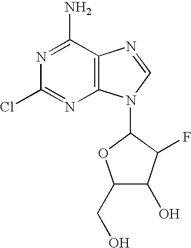 |
| CA Index Name: 9H-Purin-2-amine, 9-β-D- arabinofuranosyl-6-methoxy- Other Names: 506u; Arranon; GW 506U78; MAY; Nelarabine; Nelzarabine | 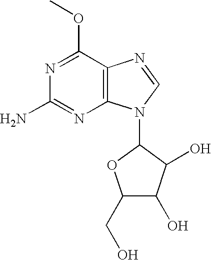 |
| CA Index Name: 2-amino-9-[2,3- bis(hydroxymethyl)cyclobutyl]-3H-purin-6- one Other Names: lobucavir | 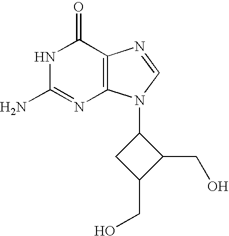 |
| CA Index Name: 2-amino-9-(2- hydroxyethoxymethyl)-3H-purin-6-one Other Names: acyclovir | 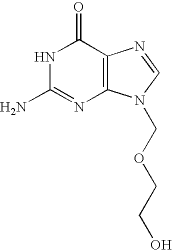 |
| CA Index Name: 2-[(2-amino-6-oxo-3,9- dihydropurin-9-yl)methoxy]ethyl-2-amino-3- methyl-butanoate Other Names: valacyclovir | 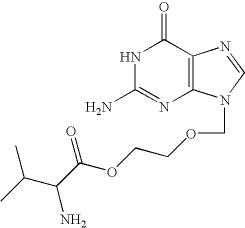 |
| CA Index Name: 2-[(2-amino-6-oxo-3,6- dihydro-9H-purin-9-yl)methoxy]-3- hydroxypropyl-2-amino-3-methyl-butanoate Other Names: valgancyclovir | 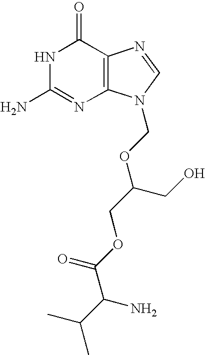 |
| CA Index Name: 2-(6-aminopurin-9-yl)-5- (hydroxymethyl)oxolane-3,4-diol hydrate Other Names: vidarabine | 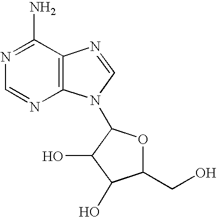 |

For convenience purposes, only one of the possible forms of the antiviral compounds of Table 2 have been shown. It is contemplated, however, that the antiviral compounds of Table 2 may have corresponding resonance structures and/or may isomerize between a variety of forms due to electron delocalization. Further, with regard to stereoisomers, it should be understood that a solid line designation for certain of the bonds as shown in the above structures for the attachment of an —H or an —OH group, or other groups, to a carbon atom of the chain or ring indicates that these groups may lie either below or above the plane of the page (i.e., [symbol] or [symbol] ). As noted elsewhere, all isomeric forms of the compounds disclosed herein are contemplated, including racemates, racemic mixtures, and individual enantiomers or diastereomers.

### Substituted or Unsubstituted Imidazoles

In addition to the antiviral agent, in various embodiments the compositions of the present invention include a substituted or unsubstituted imidazole. In general, a wide variety of substituted and unsubstituted imidazoles may be employed. The substituted or unsubstituted imidazoles that are included in the composition are generally heterocyclic aromatic compounds. The skeletal structure of the imidazoles typically includes a five-membered saturated or unsaturated ring including two nitrogen atoms separated by one carbon atom.

It has been found that combining substituted or unsubstituted imidazoles with antiviral agents corresponding to Formulae (1), (1A), and/or (2) above advantageously improves the solubility and/or increases the concentration of such antiviral agents in pharmaceutical formulations. Further, the presence of the substituted or unsubstituted imidazole enables the preparation of improved transdermal and/or transmucosal dosage forms of pharmaceutically active antiviral agent(s) (such as, for example, acyclovir and analogs, derivatives, and prodrugs thereof) by promoting the dissolution of these derivatives and/or by facilitating the dermal and/or mucosal transport of the same. Advantageously, some of the substituted and unsubstituted imidazoles disclosed herein for enhancing the solubility and/or dermal, mucosal, or other transport of pharmaceutically active antiviral compounds may also have the ability to exert their own pharmacological effect.

The improved solubility and/or increased concentration effect is evident with a variety of antiviral agents corresponding to Formulae (1), (1A), and (2) including, but not limited to, pyrimidine- and purine-based antiviral compounds such as acyclovir, brivudine, cidofovir, desciclovir, didanosine, famciclovir, fluorouracil, 2-deoxy- and 5-deoxy-5-fluorouridine, ganciclovir, idoxuridine, lamivudine, lobucavir, penciclovir, sorivudine, trifluridine, valacyclovir, valganciclovir, vidarabine, zalcitabine, zidovudine, any others listed above in Table 2, and the like, in combination with the substituted or unsubstituted imidazoles discussed in further detail below and optionally in further combination with one or more additional components.

The substituted or unsubstituted imidazole which may be included in the compositions of the present invention is an unsaturated compound generally corresponding to Formula (3):

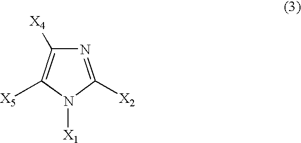

wherein:

X₁ is hydrogen, hydrocarbyl, substituted hydrocarbyl, or acyl; and

X₂, X₄, and X₅ are independently hydrogen, hydrocarbyl, substituted hydrocarbyl, heterocyclo, halo, alkoxy, amino, or nitro;

provided, however, that when the antiviral compound is a purine derivative corresponding to Formula (2) and R₉ is —CH₂OCH₂CH₂OH, then at least one of X₁, X₂, and X₅ are other than hydrogen, or X₄ is other than —CH₂—NH₂. Stating the proviso another way, when the antiviral compound corresponding to Formula (2) is acyclovir, the substituted or unsubstituted imidazole corresponding to Formula (3) is not histamine.

Although X₁ may be hydrogen, hydrocarbyl, substituted hydrocarbyl, or acyl, in certain embodiments X₁ is hydrogen, substituted or unsubstituted alkyl, substituted or unsubstituted aryl. Typically, such alkyl and aryl substituents contain from 1 to 20 carbon atoms and may be linear, branched or cyclic, and one or more hydrogen atoms of the substituted hydrocarbyl moieties are replaced with a different substituent (e.g., —OH, —OR, —COOH, —COOR, —CONH₂, —NH₂, —NHR, —NRR, —SH, —SR, —SO₂R, —SO₂H, —SOR, heterocyclo, and halo (including F, Cl, Br and I), among others, wherein each occurrence of R may be hydrocarbyl or substituted hydrocarbyl (e.g., substituted or unsubstituted alkyl, substituted or unsubstituted aryl, or substituted or unsubstituted aralkyl). In particular embodiments, X₁ is hydrogen, alkyl, aryl, —C(═O)—X₆, or —(CH₂)ₙ—X₇; wherein X₆ is hydrogen, hydroxy, hydrocarbyl, or amino; X₇ is alkoxy, alkenoxy, alkynoxy, hydroxy, halo, or amino; and n is 1, 2, 3, or 4 (subject to the above proviso). According to these embodiments, for example, X₁ may be —C(═O)—X₆, wherein X₆ is hydrogen, hydroxy, substituted alkyl, unsubstituted alkyl (e.g., methyl, ethyl, propyl, butyl, pentyl, hexyl, etc.), substituted aryl, unsubstituted aryl, or —NH₂. For example, X₆ may be hydrogen, hydroxy, methyl, ethyl, propyl, butyl, phenyl, benzyl, or —NH₂. Alternatively, according to these embodiments, X₁ may be —(CH₂)ₙ—X₇, wherein X₇ is alkoxy, alkenoxy, alkynoxy, hydroxy, halo, or amino. Thus, for example, X₁ can be an alkylene moiety (e.g., methylene, ethylene, propylene, butylene, pentylene, or hexylene) substituted with methoxy, ethoxy, ethenoxy, propenoxy, hydroxy, halo, or —NH₂.

Similarly, although X₂, X₄, and X₅ are independently hydrogen, hydrocarbyl, substituted hydrocarbyl, heterocyclo, halo, alkoxy, amino, or nitro, in certain embodiments X₂, X₄, and X₅ are independently hydrogen, substituted or unsubstituted alkyl, substituted or unsubstituted aryl, heterocyclo, halo, alkoxy, —NH₂, or nitro. Typically, such alkyl and aryl substituents contain from 1 to 20 carbon atoms and may be linear, branched or cyclic, and one or more hydrogen atoms of the substituted hydrocarbyl moieties are replaced with a different substituent (e.g., —OH, —OR, —COOH, —COOR, —CONH₂, —NH₂, —NHR, —NRR, —SH, —SR, —SO₂R, —SO₂H, —SOR, heterocyclo, and halo (including F, Cl, Br and I), among others, wherein each occurrence of R may be hydrocarbyl or substituted hydrocarbyl (e.g., substituted or unsubstituted alkyl, substituted or unsubstituted aryl, or substituted or unsubstituted aralkyl). In one particular embodiment, X₂, X₄, and X₅ are independently hydrogen, alkyl, aryl, —C(═O)—X₆, —(CH₂)ₙ—X₇, —NH₂, or —NO₃; wherein each X₆ and/or each X₇ are as defined above and each n is independently 1, 2, 3, or 4. Thus, for example, each X₆ may be independently hydrogen, hydroxy, methyl, ethyl, propyl, butyl, phenyl, benzyl, or —NH₂ and each X₇ may be independently methoxy, ethoxy, ethenoxy, propenoxy, hydroxy, halo, or —NH₂.

In combination, among certain of the preferred embodiments are substituted or unsubstituted imidazoles corresponding to Formula (3) wherein:

X₁ is hydrogen, alkyl, aryl, —C(═O)—X₆, or —(CH₂)ₙ—X₇;

X₂, X₄, and X₅ are independently hydrogen, alkyl, aryl, —C(═O)—X₆, —(CH₂)ₙ—X₇, —NH₂, or —NO₃;

each X₆ is independently hydrogen, hydroxy, methyl, ethyl, propyl, butyl, phenyl, benzyl, or —NH₂;

each X₇ is independently methoxy, ethoxy, ethenoxy, propenoxy, hydroxy, halo, or —NH₂;

and each n is independently 1, 2, 3, or 4 (subject to the above proviso).

In certain of these preferred embodiments, X₁, X₂, X₄, and X₅ may be independently hydrogen, methyl, ethyl, propyl, or butyl. In other particular embodiments, one of X₁, X₂, X₄, and X₅ is alkyl (e.g., methyl, ethyl, propyl, or butyl) and the others of X₁, X₂, X₄, and X₅ are hydrogen. In other particular embodiments, X₁ is alkyl (e.g., methyl, ethyl, propyl, or butyl) and X₂, X₄, and X₅ are each hydrogen.

Representative substituted and unsubstituted imidazoles falling within the structure of Formula (3) and the various definitions for X₁, X₂, X₄, and X₅ above include, for example, imidazole, 1-, 2-, 4-, and/or 5-methylimidazole, 1-, 2-, 4-, and/or 5-ethylimidazole, 1,2-dimethylimidazole, 1-, 2-, 4-, and/or 5-hydroxymethylimidazole, 1-, 2-, 4-, and/or 5-mercaptoimidazole, 1-, 2-, 4-, and/or 5-allylimidazole, 1-acetylimidazole, 1-, 2-, 4-, and/or 5-propylimidazole, 2-ethyl-4-methylimidazole, 1-, 2-, 4-, and/or 5-isopropylimidazole, histamine (subject, however, to the above proviso), 1-, 2-, 4-, and/or 5-imidazolecarboxylic acid, 2-, 4-, and/or 5-nitroimidazole, 2-mercapto-1-methylimidazole, 5-chloro-1-methylimidazole, 4,5-dicyanoimidazole, 1-, 2-, 4-, and/or 5-butylimidazole, 1-(3-aminopropyl)imidazole, 5-aminoimidazole-4-carboxamide, 1-, 2-, 4-, and/or 5-(2-methoxy-ethylimidazole, 4,5-dichloroimidazole, 1,2-dimethyl-5-nitroimidazole, 1-, 2-, 4-, and/or 5-hydroxyimidazole, 1-, 2-, 4-, and/or 5-phenylimidazole, 5-chloro-1-ethyl-2-methylimidazole, 1-, 2-, 4-, and/or 5-bromoimidazole, 1-, 2-, 4-, and/or 5-benzylimidazole, pharmaceutically acceptable salts thereof, combinations thereof, and the like. In a particular embodiment, the substituted or unsubstituted imidazole is selected from the group consisting of imidazole, 1-methylimidazole, 2-methylimidazole, 4-methylimidazole, a pharmaceutically acceptable salt thereof, and a combination thereof.

A pharmaceutically acceptable salt of any one or more of the substituted or unsubstituted imidazoles corresponding to Formula (3) and the various definitions for X₁, X₂, X₄, and X₅ described above, or a pharmaceutically acceptable salt of any of the particular substituted or unsubstituted imidazoles described in the preceding paragraph, may also be included in the compositions of the present invention. Suitable salts are described in further detail below. In a particular embodiment, the salt of the substituted or unsubstituted imidazole is a sodium salt (e.g., sodium imidazole).

### Non-Steroidal Anti-Inflammatory Agent

In addition to the antiviral compound (and, optionally, the substituted or unsubstituted imidazole or other component described herein), in various embodiments the compositions of the present invention include a non-steroidal anti-inflammatory agent. In general, a wide variety of non-steroidal anti-inflammatory agents may be employed, and include agents (e.g., drugs) having an analgesic, anti-pyretic, and/or anti-inflammatory affect.

It has been found that combining non-steroidal anti-inflammatory agents with antiviral compounds corresponding to Formulae (1) and (2), and in particular pyrimidine and/or purine derivatives corresponding to Formulae (1), (1A), and (2) above, advantageously improves the solubility and/or increases the concentration of such antiviral compounds in pharmaceutical formulations. Further, the presence of the non-steroidal anti-inflammatory agent enables the preparation of improved transdermal and/or transmucosal dosage forms of pharmaceutically active antiviral compounds (such as, for example, acyclovir and analogs, derivatives, and prodrugs thereof) by promoting the dissolution of these derivatives and/or by facilitating the dermal, mucosal, or other transport of the same. Advantageously, the non-steroidal anti-inflammatory agents disclosed herein for enhancing the solubility and/or dermal or mucosal transport of pharmaceutically active antiviral compounds also have the ability to exert their own pharmacological effect.

The improved solubility and/or increased concentration effect is evident with a variety of antiviral compounds including, for example, pyrimidine and/or purine derivatives corresponding to Formulae (1), (1A), and (2) including, but not limited to, acyclovir, brivudine, cidofovir, desciclovir, didanosine, famciclovir, fluorouracil, 2-deoxy- and 5-deoxy-5-fluorouridine, ganciclovir, idoxuridine, lamivudine, lobucavir, penciclovir, sorivudine, trifluridine, valacyclovir, valganciclovir, vidarabine, zalcitabine, zidovudine, any others listed in Table 2, and the like, in combination with the non-steroidal anti-inflammatory agents discussed in further detail below and in further combination with one or more additional components.

As noted above, a wide variety of non-steroidal anti-inflammatory agents and pharmaceutically acceptable salts thereof may be included in the compositions of the invention. In one embodiment, for example, the non-steroidal anti-inflammatory agent is selected from the group consisting of a salicylate derivative, an arylpropionic acid derivative (i.e., a profen), an arylbutanoic acid derivative (i.e., a bufen), a pyrazolidine derivative, an N-arylanthranilic acid derivative (i.e., a fenamic acid derivative), an oxicam, a sulfonanilide, an arylalkanoic acid derivative, an acetic acid derivative, an acetamide derivative, a pharmaceutically acceptable salt thereof, and a combination thereof.

Where the non-steroidal anti-inflammatory agent is or includes a salicylate derivative or a pharmaceutically acceptable salt thereof, the salicylate derivative may be selected from, for example, acetylsalicyclic acid, amoxiprin, benorilate, choline magnesium salicylate, choline magnesium trisalicylate, diflunisal, faislamine, magnesium salicylate, methyl salicylate, salicyclic acid, salicyl salicylate, a pharmaceutically acceptable salt thereof, and a combination thereof, among a variety of others. In one preferred embodiment, the salicylate derivative is selected from acetylsalicylic acid, salicylic acid, a pharmaceutically acceptable salt thereof, and a combination thereof.

Where the non-steroidal anti-inflammatory agent is or includes an arylpropionic acid derivative or a pharmaceutically acceptable salt thereof, an arylbutanoic acid derivative or a pharmaceutically acceptable salt thereof, or a combination thereof, the arylpropionic acid derivative and/or arylbutanoic acid derivative (or salt) may be, for example, a profen and/or a bufen. According to certain embodiments, for example, the profen and/or bufen is selected from aminoprofen, benoxaprofen, butibufen, carprofen, fenbufen, fenoprofen, flurbiprofen, ibuprofen, indoprofen, ketoprofen, ketorolac, loxoprofen, naproxen, oxaprozin, pirprofen, pranoprofen, suprofen, tiaprofenic acid, a pharmaceutically acceptable salt thereof, and a combination thereof, among a variety of others. In certain preferred embodiments, the arylpropionic acid derivative or arylbutanoic acid derivative is selected from ibuprofen, ketoprofen, flurbiprofen, a pharmaceutically acceptable salt thereof, and a combination thereof.

Where the non-steroidal anti-inflammatory agent is or includes a pyrazolidine derivative or a pharmaceutically acceptable salt thereof, the pyrazolidine derivative may be selected from, for example, azapropazone (apazone), ketophenylbutazone, metamizole, oxyphenbutazone, phenylbutazone, sulfinpyrazone, a pharmaceutically acceptable salt thereof, and a combination thereof, among a variety of others.

Where the non-steroidal anti-inflammatory agent is or includes an N-arylanthranilic acid (i.e., a fenamic acid) or a pharmaceutically acceptable salt thereof, the N-arylanthranilic acid may be selected from, for example, mefenamic acid, meclofenamic acid, flufenamic acid, a pharmaceutically acceptable salt thereof, and a combination thereof, among a variety of others.

Where the non-steroidal anti-inflammatory agent is or includes an oxicam or a pharmaceutically acceptable salt thereof, the oxicam may be selected from, for example, isoxicam, lornoxicam, meloxicam, piroxicam, tenoxicam, a pharmaceutically acceptable salt thereof, and a combination thereof, among a variety of others.

Where the non-steroidal anti-inflammatory agent is or includes an arylalkanoic acid or a pharmaceutically acceptable salt thereof, the arylalkanoic acid may be selected from, for example, diclofenac, ibufenac, aceclofenac, bromfenac, etodolac, indomethacin, nalbumetone, sulindac, tolmetin, zomepirac, a pharmaceutically acceptable salt thereof, and a combination thereof, among a variety of others. In certain preferred embodiments, the arylalkanoic acid is selected from diclofenac, sulindac, a pharmaceutically acceptable salt thereof, and a combination thereof.

Other suitable non-steroidal anti-inflammatory agents which may be included in these or other embodiments include, for example, sulfonanilides such as nimesulide and acetamide derivatives such as difenpiramide.

As noted above, a pharmaceutically acceptable salt of any one or more of the non-steroidal anti-inflammatory agents discussed above may also be included in the compositions of the present invention. In a particular embodiment, the salt is an imidazolium salt. In another particular embodiment, the salt is a hydrochloric acid salt, a sodium salt, or a potassium salt. Other suitable salts are described in further detail below.

### Additional Acids or Salts Thereof

In addition to the antiviral compound (and in certain embodiments in addition to the substituted or unsubstituted imidazole and/or the non-steroidal anti-inflammatory agent or other component described herein), in various embodiments the compositions of the present invention include one or more acids or pharmaceutically acceptable salts thereof, including amino, carboxylic, or sulfonic acids or salts or esters thereof. In a particular embodiment, the composition includes an amino acid or a pharmaceutically acceptable salt thereof, a carboxylic acid or a pharmaceutically acceptable salt thereof, a sulfonic acid or a pharmaceutically acceptable salt thereof, and combinations thereof.

Where the acid or salt thereof is a carboxylic acid and/or a sulfonic acid, for example, the carboxylic acid and sulfonic acid generally correspond to the formulae: Z₁-C(═O)—OH and Z₁-S(═O)₂—OH, respectively, wherein Z₁ is hydrocarbyl or substituted hydrocarbyl. Typically, such hydrocarbyl substituents (i.e., Z₁) contain from 1 to 20 carbon atoms and may be linear, branched, or cyclic, non-aromatic or aromatic groups (i.e., an aromatic carboxylic acid); thus, for example, the Z₁ substituent may be selected from methyl, ethyl, n-propyl, cyclopropyl, isopropyl, n-butyl, cyclobutyl, isobutyl, s-butyl, n-pentyl, isopentyl, cyclopentyl, n-hexyl, isohexyl, cyclohexyl, benzyl, phenyl, and napthyl. Similar to the hydrocarbyl moieties described in the preceding sentence, the substituted hydrocarbyl moieties (i.e., Z₁) may contain 1 to 20 carbon atoms and may be linear, branched, or cyclic; one or more hydrogen atoms of the substituted hydrocarbyl moieties, however, are replaced with a different substituent such as, for example, —OH, —OR, —COOH, —COOR, —CONH₂, —NH₂, —NHR, —NRR, —SH, —SR, —SO₂R, —SO₂H, —SOR, heterocyclo, and halo (including F, Cl, Br and I), among others, wherein each occurrence of R may be hydrocarbyl or substituted hydrocarbyl (e.g., substituted or unsubstituted alkyl, substituted or unsubstituted aryl, or substituted or unsubstituted aralkyl). Such substituted hydrocarbyl-substituted carboxylic acids may correspond to particular amino acids, or the amino acid may be an alternative or unnatural amino acid as described elsewhere herein.

Among other representative aryl- and alkyl-carboxylic acids and salts thereof and aryl- and alkyl-sulfonic acids and salts thereof, exemplary carboxylic and sulfonic acids (and salts thereof) that may be included in the compositions of the invention include naphthalenesulfonic acid, lipoic acid, benzoic acid, 2-acetoxybenzoic acid (i.e., aspirin), 5-aminosalicylic acid, sulfanilic acid, pharmaceutically acceptable salts thereof, and combinations thereof.

The acid or salt thereof included in the compositions of the invention may additionally or alternatively be an amino acid or a pharmaceutically acceptable salt thereof. For example, the amino acid or salt thereof may be one or more of the twenty standard amino acids noted above (i.e., alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and/or valine). Additionally or alternatively, the amino acid or salt thereof may correspond to a non-naturally occurring amino acid. As noted above, the non-naturally occurring amino acids include esters of proteogenic L-amino acids (i.e., from the 20 amino acids commonly incorporated into proteins), as well as esters of D-amino acids and esters of non-proteogenic amino acids (i.e., metabolites or analogs of proteogenic amino acids) (e.g., diketopiperazines (i.e., cyclic anhydrides of two amino acids)), among other species.

As noted above, a pharmaceutically acceptable salt of any one or more of the carboxylic, sulfonic, and/or amino acids, may also be included in the compositions of the present invention. In a particular embodiment, the salt is an imidazolium salt; according to this embodiment, therefore, the additional component included in the composition is selected from an imidazolium salt of an amino acid, an imidazolium salt of a carboxylic acid, an imidazolium salt of a sulfonic acid, and combinations thereof. Other suitable salts are described in further detail below.

### Pharmaceutically Acceptable Carriers

In addition to the various species discussed above (i.e., the antiviral compound(s) (e.g., pyrimidine and/or purine derivatives), the substituted or unsubstituted imidazole, the non-steroidal anti-inflammatory agent, and the acids) the composition may include a pharmaceutically acceptable carrier. The carrier, also known in the art as an excipient, vehicle, auxiliary, adjuvant, or diluent, is typically a substance which is pharmaceutically inert, confers a suitable consistency or form to the composition, and does not diminish the efficacy of the composition. The carrier is generally considered to be pharmaceutically or pharmacologically acceptable if it does not produce an unacceptably adverse, allergic or other untoward reaction when administered to a mammal, especially a human.

Pharmaceutically acceptable carriers for use in the compositions of the present invention are well known to those of ordinary skill in the art and are selected based upon a number of factors: the particular compound(s) used in the composition, and its concentration, stability and intended bioavailability; the subject, its age, size and general condition; and the route of administration. Suitable nonaqueous, pharmaceutically-acceptable solvents include, but are not limited to, water, alcohols (e.g., α-glycerol formal, β-glycerol formal, 1,3-butyleneglycol, aliphatic or aromatic alcohols having 2-30 carbon atoms such as methanol, ethanol, propanol, isopropanol, butanol, t-butanol, hexanol, octanol, amylene hydrate, benzyl alcohol, glycerin (glycerol), glycol, hexylene glycol, tetrahydrofurfuryl alcohol, menthol (e.g., 1-menthol), lauryl alcohol, cetyl alcohol, or stearyl alcohol, fatty acid esters of fatty alcohols such as polyalkylene glycols (e.g., polypropylene glycol, polyethylene glycol), sorbitan, sucrose and cholesterol); amides (e.g., dimethylacetarnide (DMA), benzyl benzoate DMA, dimethylformamide, N-(β-hydroxyethyl)-lactamide, or N,N-dimethylacetamide amides); esters (e.g., acetate esters such as monoacetin, diacetin, and triacetin, aliphatic or aromatic esters such as ethyl caprylate or octanoate, alkyl oleate, benzyl benzoate, benzyl acetate, dimethylsulfoxide (DMSO), esters of glycerin such as mono, di, or tri-glyceryl citrates or tartrates, ethyl benzoate, ethyl acetate, ethyl carbonate, ethyl lactate, ethyl oleate, fatty acid esters of sorbitan, fatty acid derived PEG esters, glyceryl monostearate, glyceride esters such as mono, di, or tri-glycerides, fatty acid esters such as isopropyl myristrate, fatty acid derived PEG esters such as PEG-hydroxyoleate and PEG-hydroxystearate, pluronic 60, polyoxyethylene sorbitol oleic polyesters such as poly(ethoxylated)₃₀₋₆₀ sorbitol poly(oleate)₂₋₄, poly(oxyethylene)₁₅₋₂₀ monooleate, poly(oxyethylene)₁₅₋₂₀ mono 12-hydroxystearate, and poly(oxyethylene)₁₅₋₂₀ mono ricinoleate, polyoxyethylene sorbitan esters such as polyoxyethylene-sorbitan monooleate, polyoxyethylene-sorbitan monopalmitate, polyoxyethylene-sorbitan monolaurate, polyoxyethylene-sorbitan monostearate, and Polysorbate® 20, 40, 60 or 80 from ICI Americas, Wilmington, Del., alkyleneoxy modified fatty acid esters such as polyoxyl 40 hydrogenated castor oil and polyoxyethylated castor oils (e.g., Cremophor® EL solution or Cremophor® RH 40 solution), saccharide fatty acid esters (i.e., the condensation product of a monosaccharide (e.g., pentoses such as ribose, ribulose, arabinose, xylose, lyxose and xylulose, hexoses such as glucose, fructose, galactose, mannose and sorbose, trioses, tetroses, heptoses, and octoses), disaccharide (e.g., sucrose, maltose, lactose and trehalose) or oligosaccharide or mixture thereof with a C₄-C₂₂ fatty acid(s) (e.g., saturated fatty acids such as caprylic acid, capric acid, lauric acid, myristic acid, palmitic acid and stearic acid, and unsaturated fatty acids such as palmitoleic acid, oleic acid, elaidic acid, erucic acid and linoleic acid)), or steroidal esters); mono- and dialkyl sulfoxides such as DMSO and decylmethylsulfoxide; alkyl, aryl, or cyclic ethers having 2-30 carbon atoms (e.g., diethyl ether, tetrahydrofuran, dimethyl isosorbide, diethylene glycol monoethyl ether); glycofurol (tetrahydrofurfuryl alcohol polyethylene glycol ether); ketones having 3-30 carbon atoms (e.g., acetone, methyl ethyl ketone, methyl isobutyl ketone); aliphatic, cycloaliphatic or aromatic hydrocarbons having 4-30 carbon atoms (e.g., benzene, cyclohexane, dichloromethane, dioxolanes, hexane, n-decane, n-dodecane, n-hexane, sulfolane, tetramethylenesulfone, tetramethylenesulfoxide, toluene, dimethylsulfoxide (DMSO), or tetramethylenesulfoxide); oils of mineral, vegetable, animal, essential or synthetic origin (e.g., mineral oils such as aliphatic or wax-based hydrocarbons, aromatic hydrocarbons, mixed aliphatic and aromatic based hydrocarbons, and refined paraffin oil, vegetable oils such as linseed, tung, safflower, soybean, castor, cottonseed, groundnut, rapeseed, coconut, palm, olive, corn, corn germ, sesame, persic and peanut oil and glycerides such as mono-, di- or triglycerides, animal oils such as fish, marine, sperm, cod-liver, haliver, squalene, squalane, and shark liver oil, oleic oils, and polyoxyethylated castor oil); alkyl or aryl halides having 1-30 carbon atoms and optionally more than one halogen substituent; methylene chloride; monoethanolamine; petroleum benzin; trolamine; omega-3 polyunsaturated fatty acids (e.g., alpha-linolenic acid, eicosapentaenoic acid, docosapentaenoic acid, or docosahexaenoic acid); polyglycol ester of 12-hydroxystearic acid and polyethylene glycol (Solutol® HS-15, from BASF, Ludwigshafen, Germany); polyoxyethylene glycerol; sodium laurate; sodium oleate; or sorbitan monooleate.

Other pharmaceutically acceptable solvents for use in the invention are well known to those of ordinary skill in the art, and are identified in The Chemotherapy Source Book (Williams & Wilkens Publishing), The Handbook of Pharmaceutical Excipients, (American Pharmaceutical Association, Washington, D.C., and The Pharmaceutical Society of Great Britain, London, England, 1968), Modern Pharmaceutics, (G. Banker et al., eds., 3d ed.) (Marcel Dekker, Inc., New York, N.Y., 1995), The Pharmacological Basis of Therapeutics, (Goodman & Gilman, McGraw Hill Publishing), Pharmaceutical Dosage Forms, (H. Lieberman et al., eds.) (Marcel Dekker, Inc., New York, N.Y., 1980), Remington's Pharmaceutical Sciences (A. Gennaro, ed., 19th ed.) (Mack Publishing, Easton, Pa., 1995), The United States Pharmacopeia 24, The National Formulary 19, (National Publishing, Philadelphia, Pa., 2000), A. J. Spiegel et al., and Use of Nonaqueous Solvents in Parenteral Products, Journal of Pharmaceutical Sciences, Vol. 52, No. 10, pp. 917-927 (1963).

Any one or more of the above-listed pharmaceutically acceptable carriers may also function as an additional penetration enhancer in the composition (e.g., for transdermal and/or transmucosal application), or conventional penetration enhancers may be additionally or alternatively included in the composition. Various penetration enhancers are known, and include, for instance, water, low molecular weight alcohols (e.g., methanol, ethanol, propanol, isopropanol, t-butanol, and the like), alkyl methyl sulfoxides (e.g., decylmethylsulfoxide (DecMSO)), non-ionic surfactants (e.g., polysorbates, polyethoxylated alkyl ethers and esters and poloxamers, oleic acid, propylene glycol, Azone® (also known as I -dodecylazacycloheptan-2-one or laurocapram), aprotic dipolar solvents (such as dimethylformamide, dimethylsulfoxide, dimethylacetamide, and the like), capsaicin and analogs thereof (e.g., nonivamide), menthol (e.g., 1-menthol), caffeine, combinations thereof, and the like.

Where a penetration enhancer, a pharmaceutically acceptable carrier or a combination thereof is included in the composition, it is typically included at a concentration of from about 1 wt. % to about 90 wt. %. Thus, for example, the penetration enhancer and/or the pharmaceutically acceptable carrier may be included in the composition at a concentration of about 5 wt. %, about 10 wt. %, about 15 wt. %, about 20 wt. %, about 25 wt. %, about 30 wt. %, about 40 wt. %, about 50 wt. %, about 60 wt. %, or about 75 wt. %. In a particular embodiment, the penetration enhancer and/or the pharmaceutically acceptable carrier is included in the composition at a concentration of from about 10 wt. % to about 50 wt. %.

It will be understood that the inclusion of other excipients in the composition may also be useful. These excipients may be utilized with the antiviral compound, substituted or unsubstituted imidazole, non-steroidal anti-inflammatory agent and/or amino, carboxylic or sulfonic acid mixture in order to formulate the mixture into aerosols, creams, emulsions, foams, gels/jellies, lotions, ointments, pastes, soaps, solutions, sprays, suspensions, tinctures, transdermal patches, and the like. The antiviral compound, substituted or unsubstituted imidazole, and/or non-steroidal anti-inflammatory agent can be added to other dosage form ingredients in essentially any manner that does not substantially alter the antiviral compound, substituted or unsubstituted imidazole, non-steroidal anti-inflammatory agent, and/or amino, carboxylic or sulfonic acid, or other components, and/or the beneficial effects thereof.

### Additional Pharmaceutically Active Agents

In addition to the species discussed above (i.e., the antiviral compound(s) (e.g., pyrimidine and/or purine derivatives), the substituted or unsubstituted imidazole, the non-steroidal anti-inflammatory agent, and/or amino, carboxylic or sulfonic acid), the compositions of the present invention may also include one or more additional pharmaceutically active components. Suitable pharmaceutically active agents that may be included in the compositions of the present invention include, for instance, anesthetics, antihypertensives, antianxiety agents, anticlotting agents, anticonvulsants, blood glucose-lowering agents, decongestants, antihistamines, antitussives, antineoplastics, beta blockers, anti-inflammatory agents (other than those noted above), antipsychotic agents, cognitive enhancers, cholesterol-reducing agents, antiobesity agents, autoimmune disorder agents, anti-impotence agents, antibacterial and antifungal agents, hypnotic agents, anti-Parkinsonism agents, anti-Alzheimer's Disease agents, antibiotics, anti-depressants, and antiviral agents (other than those noted above).

Specific examples of the above and other classes of drugs and therapeutic agents that may be included in the compositions of the present invention are set forth below, by way of example only. Each named drug should be understood to include the neutral form of the drug, pharmaceutically acceptable salts, as well as prodrugs. Specific examples of antihypertensives include prazosin, nifedipine, trimazosin and doxazosin; a specific example of an antianxiety agent is hydroxyzine; a specific example of a blood glucose-lowering agent is glipizide; a specific example of an anti-impotence agent is sildenafil citrate; specific examples of antineoplastics include chlorambucil, lomustine and echinomycin; a specific example of an imidazole-type antineoplastic is tubulazole; a specific example of a barbiturate is phenobarbital; specific examples of vitamins/nutritional agents include retinol and vitamin E; specific examples of beta blockers include timolol and nadolol; a specific example of an emetic is apomorphine; specific examples of a diuretic include chlorthalidone and spironolactone; a specific example of an anticoagulant is dicumarol; specific examples of cardiotonics include digoxin and digitoxin; specific examples of androgens include 17-methyltestosterone and testosterone; a specific example of a mineral corticoid is desoxycorticosterone; a specific example of a steroidal hypnotic/anesthetic is alfaxalone; specific examples of anabolic agents include fluoxymesterone and methanstenolone; specific examples of antidepression agents include sulpiride, [3,6-dimethyl-2-(2,4,6-trimethyl-phenoxy)-pyridin-4-yl]-(1-ethylpropyl)-amine, 3,5-dimethyl-4-(3′-pentoxy)-2-(2′,4′,6′-trimethylphenoxy) pyridine, pyroxidine, fluoxetine, paroxetine, venlafaxine and sertraline; specific examples of antibiotics include ampicillin and penicillin G; specific examples of anti-infectives include benzalkonium chloride and chlorhexidine; specific examples of coronary vasodilators include nitroglycerin and mioflazine; a specific example of a hypnotic is etomidate; specific examples of carbonic anhydrase inhibitors include acetazolamide and chlorzolamide; specific examples of antifungals include econazole, terconazole, fluconazole, voriconazole, and griseofulvin; a specific example of an antiprotozoal is metronidazole; specific examples of anthelmintic agents include tiabendazole and oxfendazole and morantel; specific examples of antihistamines include astemizole, levocabastine, cetirizine, and cinnarizine; specific examples of antipsychotics include ziprasidone, fluspirilene, risperidone and penfluridole; specific examples of gastrointestinal agents include loperamide and cisapride; specific examples of serotonin antagonists include ketanserin and mianserin; specific examples of anesthetics include benzocaine, chloroprocaine, cocaine, procaine, tetracaine, bupivacaine, levobupivacaine, lidocaine, mepivacaine, prilocaine, ropivacaine, articaine, trimecaine, and combinations thereof; a specific example of a hypoglycemic agent is acetohexamide; a specific example of an anti-emetic is dimenhydrinate; a specific example of an antibacterial is cotrimoxazole; a specific example of a dopaminergic agent is L-DOPA; specific examples of anti-Alzheimer's Disease agents are THA and donepezil; a specific example of an anti-ulcer agent/H2 antagonist is famotidine; specific examples of sedative/hypnotic agents include chlordiazepoxide and triazolam; a specific example of a vasodilator is alprostadil; a specific example of a platelet inhibitor is prostacyclin; specific examples of ACE inhibitor/antihypertensive agents include enalaprilic acid and lisinopril; specific examples of tetracycline antibiotics include oxytetracycline and minocycline; specific examples of macrolide antibiotics include erythromycin, azithromycin, clarithromycin, and spiramycin; specific examples of glycogen phosphorylase inhibitors include [R, S]-5-chloro-N-[2-hydroxy-3-[methoxymethylamino]-3-oxo-1-(phenylmethyl)propyl-1H-indole-2-carboxamide and 5-chloro-1H-indole-2-carboxylic acid [(1S)-benzyl-(2R)-hydroxy-3-((3R, 4S)-dihydroxy-pyrrolidin-1-yl-)-3-oxypropyl]amide.

Further examples of drugs that may be included in the compositions of the present invention are the glucose-lowering drug chlorpropamide, the anti-fungal fluconazole, the anti-hypercholesterolemic atorvastatin calcium, the antipsychotic thiothixene hydrochloride, the anxiolytics hydroxyzine hydrochloride and doxepin hydrochloride, the anti-hypertensive amlodipine besylate, the anti-inflammatory agents piroxicam, valdicoxib, carprofen, and celicoxib, and the antibiotics carbenicillin indanyl sodium, bacampicillin hydrochloride, troleandomycin, and doxycycline hyclate.

### Pharmaceutically Acceptable Salts

As noted above, the various species present in the compositions of the invention (i.e., the antiviral compound(s) (e.g., pyrimidine and/or purine derivatives), the substituted or unsubstituted imidazole, the non-steroidal anti-inflammatory agent, and the acid) may be independently used as their neutral (e.g., free base) or acidic form or as their pharmaceutically acceptable salts, which includes their therapeutically-, prophylactically-, dermatologically-, medically-, and/or cosmetically-acceptable salts. Thus, reference to the antiviral compound, pyrimidine derivative, the purine derivative, the substituted or unsubstituted imidazole, and/or any other pharmaceutically active agent will, by definition, embrace the salts thereof as well (e.g., sodium imidazole). Such salts may be prepared from pharmaceutically and chemically acceptable non-toxic acids or bases, including inorganic and organic acids and inorganic and organic bases. Suitable non-toxic acids include inorganic and organic acids of basic residues such as amines, for example, acetic, benzenesulfonic, benzoic, 2-hydroxybenzoic, amphorsulfonic, citric, fumaric, gluconic, glutamic, hydrobromic, hydrochloric, isethionic, lactic, maleic, malic, mandelic, methanesulfonic, mucic, nitric, pamoic, pantothenic, phosphoric, pyroglutamic, succinic, sulfuric, barbaric acid, p-toluenesulfonic and the like; and alkali or organic salts of acidic residues such as carboxylic acids, for example, alkali and alkaline earth metal salts derived from the following bases: sodium hydride, sodium hydroxide, potassium hydroxide, calcium hydroxide, lithium hydroxide, magnesium hydroxide, and zinc hydroxide, and non-alkali salts derived from the following bases: ammonia, trimethylammonia, triethylammonia, ethylenediamine, lysine, arginine, ornithine, choline, N,N″-dibenzylethylenediamine, chloroprocaine, diethanolamine, procaine, n-benzylphenethylamine, diethylamine, piperazine, tris(hydroxymethyl)-aminomethane, tetramethylammonium hydroxide; and substituted or unsubstituted imidazoles. Pharmaceutically acceptable salts of the pyrimidine derivative, the purine derivative, the substituted or unsubstituted imidazole, or another pharmaceutically active agent, for example, can be prepared by reacting the free acid or base form of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. In addition to the anions and cations noted above, lists of suitable salts are found in Remington's Pharmaceutical Sciences, 17th ed., Mack Publishing Company, Easton, Pa., p. 1418, the disclosure of which is hereby incorporated by reference herein. In a particular embodiment, the salt for one or more of the antiviral compound, the substituted or unsubstituted imidazole, the non-steroidal anti-inflammatory agent, and the acid (e.g., amino, carboxylic, or sulfonic acid) is an imidazolium salt.

If the various compounds included in the composition of the present invention (e.g., the antiviral compound corresponding to Formulae (1), (1A), and (2), the substituted or unsubstituted imidazole corresponding to Formulae (3), the non-steroidal anti-inflammatory agent, acid, or other pharmaceutically active agent (if present)) contain more than one group which is capable of forming pharmaceutically acceptable salts, the present invention is intended to encompass multiple salts. Typical multiple salt forms include, for instance, bitartrate, diacetate, difumarate, dimeglumine, diphosphate, disodium, and trihydrochloride.

Compounds included in the compositions of the present invention (e.g., the antiviral compound corresponding to Formulae (1), (1A), and (2), the substituted or unsubstituted imidazole corresponding to Formulae (3), the non-steroidal anti-inflammatory agent, acid, or other pharmaceutically active agent (if present)) which contain basic nitrogen-containing groups may be quaternized using various quaternizing agents such as, for example, C₁-C₄ alkyl and alkenyl halides (e.g., allyl, methyl, ethyl, propyl, isopropyl, and tert-butyl chlorides, bromides, and iodides); dialkyl sulfates (e.g., dimethyl, diethyl, and diamyl sulfates); C₅-C₁₈ alkyl halides (e.g., decyl, dodecyl, lauryl, myristyl, and stearyl chlorides, bromides, and iodides); aryl-(C₁-C₄)-alkyl halides (e.g., benzyl chloride and phenethyl bromide), and the like. Such quaternary salts can assist in the preparation of both water-soluble and oil-soluble compounds.

### Prodrugs

Since prodrugs are known to enhance numerous desirable pharmaceuticals (e.g., by way of solubility, bioavailability, manufacturing), the species discussed above (i.e., the antiviral compound(s) (e.g., pyrimidine and/or purine derivatives), the substituted or unsubstituted imidazole, the non-steroidal anti-inflammatory agent, acid, or other compound or drug) may be delivered in prodrug form. Thus, the present invention is intended to cover prodrugs of pyrimidine- and/or purine-based compounds corresponding to Formulae (1), (1A), and (2), prodrugs of non-steroidal anti-inflammatory agents, methods of delivering the same, and compositions containing them. Prodrugs generally include any covalently bonded carriers which release an active parent drug in vivo when such prodrug is administered to a mammalian subject. Prodrugs are generally prepared by modifying functional groups present in the compound in such a way that the modifications are cleaved, either in routine manipulation or in vivo, to the parent compound. Prodrugs include compounds of the present invention wherein a hydroxy or amino group is bonded to any group that, when the prodrug is administered to a mammalian subject, cleaves to form a free hydroxy or free amino group, respectively. Examples of prodrugs include, but are not limited to, acetate, formate, and benzoate derivatives of alcohol and amine functional groups in the compounds and conjugates included in the compositions of the present invention. Prodrugs of the pyrimidine- and/or purine-based compounds corresponding to Formulae (1), (1A), and (2) and the non-steroidal anti-inflammatory agents discussed above are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the invention. Prodrugs may refer to compounds that are rapidly transformed in vivo to yield the pyrimidine- and/or purine-based compounds, for example, by hydrolysis in blood. A thorough discussion of prodrugs is provided in the following: Design of Prodrugs, H. Bundgaard, ea., Elsevier, 1985; Methods in Enzymology, K. Widder et al, Ed., Academic Press, 42, p. 309-396, 251985; A Textbook of Drug Design and Development, Krogsgaard-Larsen and H. Bundgaard, ea., Chapter 5; “Design and Applications of Prodrugs” p. 113-191, 1991; Advanced Drug Delivery Reviews, H. Bundgard, 8, p. 1-38, 1992; Journal of Pharmaceutical Sciences, 77, p. 285, 30 1988; Chem. Pharm. Bull., N. Nakeya et al, 32, p. 692, 1984; Pro-drugs as Novel Delivery Systems, T. Higuchi and V. Stella, Vol. 14 of the A.C.S. Symposium Series, and Bioreversible Carriers in Drug Design, Edward B. Roche, ea., American Pharmaceutical Association and Pergamon Press, 1987, each of which is hereby incorporated by reference herein.

### Tautomers, Geometric Isomers, and Stereoisomers

The compositions of the present invention may include compounds (e.g., the pyrimidine and/or purine derivative corresponding to Formulae (1), (1A), and (2), respectively, the substituted or unsubstituted imidazole corresponding to Formula (3), the non-steroidal anti-inflammatory agent, or other pharmaceutically active agent (if present)) which can exist in tautomeric, geometric or stercoisomeric forms. The present invention contemplates all such compounds, including cis- and trans-geometric isomers, E- and Z-geometric isomers, R- and S-enantiomers, diastereomers, d-isomers, 1-isomers, the racemic mixtures thereof and other mixtures thereof, as falling within the scope of the invention. Pharmaceutically acceptable salts of such tautomeric, geometric or stereoisomeric forms are also included within the invention. The terms “cis” and “trans” denote a form of geometric isomerism in which two carbon atoms connected by a double bond will each have a hydrogen atom on the same side of the double bond (“cis”) or on opposite sides of the double bond (“trans”). Some of the compounds described contain alkenyl groups, and are meant to include both cis and trans or “E” and “Z” geometric forms. Some of the compounds described contain one or more stereocenters and are meant to include R, S, and mixtures of R and S forms for each stereocenter present. Additionally, the compounds described and illustrated above may have one or more resonance structures and/or may isomerize between a variety of forms due to electron delocalization. It will be understood that the additional forms of such compounds are within the scope of the invention, but a recitation of each possible form is not included in the interest of brevity.

### Isotopes

The compositions of the present invention may also include compounds (e.g., the pyrimidine and/or purine derivative corresponding to Formulae (1), (1A), and (2), respectively, the substituted or unsubstituted imidazole corresponding to Formula (3), the non-steroidal anti-inflammatory agent, or other pharmaceutically active agent (if present)) containing unnatural proportions of atomic isotopes at one or more of the atoms that constitute such compounds. Examples of isotopes which are readily commercially available and which can be incorporated into one or more compounds included in the compositions of the present invention include isotopes of hydrogen, carbon, nitrogen, oxygen, phosphorous, sulfur, fluorine, and chlorine, for example, ²H, ³H, ¹³C, ¹⁴C, ¹⁵N, ¹⁷O, ¹⁸O, ³¹P, ³²P, ³⁵S, ¹⁸F, and ³⁶C, respectively. All isotopic variations of the compounds described herein, whether radioactive or not, are intended to be included within the scope of the present invention.

### Methods for Prophylaxis or Treatment

Another aspect of the present invention is a method for the prophylaxis or treatment of certain diseases, pathological disorders, and medical conditions. The methods generally involve administering to a mammal in need of such prophylaxis or treatment a prophylactic or therapeutic amount of the composition described above (e.g., a composition comprising: (A) an antiviral compound or a pharmaceutically acceptable salt thereof; and (B) an agent selected from the group consisting of a substituted or unsubstituted imidazole or a pharmaceutically acceptable salt thereof; a non-steroidal anti-inflammatory agent or a pharmaceutically acceptable salt thereof; an amino acid or a pharmaceutically acceptable salt thereof; a carboxylic acid or a pharmaceutically acceptable salt thereof; a sulfonic acid or a pharmaceutically acceptable salt thereof; and a combination thereof). The compositions employed in the methods of treatment and prophylaxis described herein can be therapeutic, prophylactic, dermatological, pharmaceutical, medical, and/or cosmetic compositions, depending on the particular application for which it is to be used.

In general, methods for the treatment of any disease, pathological disorder, or medical condition which will favorably respond to the compositions and methods described herein are contemplated. In addition, the compositions may also be administered to a mammal as a prophylactic measure; that is, the mammal is in need of treatment to prevent or delay the occurrence or onset of a disease, pathological disorder, or medical condition.

The above-described compositions are suitable in methods for the treatment or prophylaxis of infections such as viral, bacterial, or fungal infections. Particular aspects of the present invention, therefore, are directed to topical pharmaceutical compositions. In a particular embodiment, the method is for the treatment or prophylaxis of a viral infection; more preferably in this embodiment the method is for the treatment of a DNA virus in which the virus is sensitive to up-regulation of TNF-α in its host so that the viral replication or other vital activities are adversely affected by the above-described compositions and components thereof. Such viruses and viral infections include, for example, those selected from herpes simplex virus type 1 (HSV-1), herpes simplex virus type 2 (HSV-2), cytomegalovirus (CMV), varicella zoster virus (VZV) (i.e., shingles), Epstein-Barr Virus (EBV), human herpes virus type 6 (HHV-6), human herpes virus type 7 (HHV-7), human herpes virus type 8 (HHV-8), HIV-1, HIV-2, and HIV-3, influenza, adenoviruses, and the like. As noted above, several pyrimidine- and purine-based pharmaceutically active compounds corresponding to Formulae (1), (1A), and (2) above (such as, for instance, acyclovir, famciclovir, desciclovir, penciclovir, zidovudine, ganciclovir, didanosine, zalcitabine, valacyclovir, sorivudine, lobucavir, brivudine, cidofovir, vidarabine, idoxuridine, trifluridine, lamivudine, valganciclovir, and any others listed in Table 2 or generally corresponding to Formulae (1), (1A), and (2) and the various definitions for -A-B—, R₁, R₂, R₅, R_{6,} and R₉ above) are known to provide a prophylactic and/or therapeutic effect when administered to mammals afflicted (or subject to affliction, in the case of prophylaxis) with one or more of these viral infections.

The compositions of the present invention are also suitable in methods for the treatment or prophylaxis of benign and malignant tumors/neoplasia including cancer, such as colorectal cancer, brain cancer, bone cancer, epithelial cell-derived neoplasia (epithelial carcinoma) such as basal cell carcinoma, adenocarcinoma, gastrointestinal cancer such as lip cancer, mouth cancer, esophogeal cancer, small bowel cancer and stomach cancer, colon cancer, liver cancer, bladder cancer, pancreatic cancer, ovarian cancer, cervical cancer, lung cancer, breast cancer and skin cancer, such as squamus cell and basal cell cancers, prostate cancer, renal cell carcinoma, and other known cancers that affect epithelial cells throughout the body. Several pyrimidine- and/or purine-based pharmaceutically active compounds corresponding to Formulae (1), (1A), and (2) above (such as, for instance, fluorouracil, fluorouridine, and others corresponding to Formulae (1), (1A), and (2) and the various definitions of -A-B—, R₁, R₂, R₅, R_{6,} and R₉ above), for example, are known to provide antitumor effects when administered to a mammal.

### Dosage/Amount of the Composition Administered and Time Course of Treatment

The dose or amount of the composition administered to the mammal should be an effective amount for the intended purpose, i.e., treatment or prophylaxis of the disease, pathological disorder, or medical condition. Generally speaking, the effective amount of the composition administered to the mammal can vary according to a variety of factors such as, for example, the age, weight, sex, diet, route of administration, and the medical condition of the mammal. Specifically preferred doses are discussed more fully below. It will be understood, however, that the total daily usage of the compositions described herein will be decided by the attending physician or veterinarian within the scope of sound medical judgment.

The specific therapeutically effective dose level for any particular mammal will depend upon a variety of factors including the disorder being treated and the severity of the disorder; activity of the specific compound(s) employed; the age, body weight, general health, sex and diet of the patient; the time of administration; the route of administration; the rate of excretion of the specific compound(s) employed; the duration of the treatment; drugs used in combination or coincidental with the specific compound(s) employed and like factors well known in the medical and/or veterinary arts. For example, it is well within the skill of the art to start doses of the compound(s) at levels lower than those required to achieve the desired therapeutic effect and to gradually increase the dosage until the desired effect is achieved. If desired, the effective daily doses may be divided into multiple doses for purposes of administration. Consequently, single dose compositions may contain such amounts or submultiples to make up the daily dose.

Administration of the compositions can occur as a single event or over a time course of treatment. For example, one or more of the compositions can be administered hourly (e.g., every hour, every two hours, every three hours, every four hours, every five hours, every six hours, and so on), daily, weekly, bi-weekly, or monthly. For treatment of acute conditions, the time course of treatment may be at least several hours or days. Certain conditions could extend treatment from several days to several weeks. For example, treatment could extend over one week, two weeks, or three weeks. For more chronic conditions, treatment could extend from several weeks to several months, a year or more, or the lifetime of the mammal in need of such treatment. Alternatively, the compositions can be administered hourly, daily, weekly, bi-weekly, or monthly, for a period of several weeks, months, years, or over the lifetime of the mammal as a prophylactic measure.

The compositions of the present invention may also be co-administered, meaning that the antiviral compound can be administered separately from, but within the same general time frame as, the substituted or unsubstituted imidazole and/or the non-steroidal anti-inflammatory agent. Thus, the antiviral agent can, for example, be administered in its own dosage form which is administered at approximately the same time as the substituted or unsubstituted imidazole and/or the non-steroidal anti-inflammatory agent, which is/are in a separate dosage form(s). If administered separately, it is generally preferred to administer the antiviral agent and the substituted or unsubstituted imidazole and/or the non-steroidal anti-inflammatory agent within a matter of minutes of each other, so that the two are present together in the environment of use. Preferably, the antiviral compound (e.g., the pyrimidine and/or the purine derivative) and the substituted or unsubstituted imidazole and/or the non-steroidal anti-inflammatory agent or amino, carboxylic or sulfonic acid are administered simultaneously or substantially simultaneously.

### Routes of Administration

As noted above, the selection of the above-described pharmaceutically acceptable carrier can, in part, be a function of the route of administration. In general, the compositions of the present invention can be formulated for any route of administration so long as the blood circulation system is available via that route. For example, suitable routes of administration include, but are not limited to, oral, parenteral (e.g., intravenous, intraarterial, subcutaneous, rectal, subcutaneous, intramuscular, intraorbital, intracapsular, intraspinal, intraperitoneal, or intrasternal), topical (nasal, transdermal, intraocular), intravesical, intrathecal, enteral, pulmonary, intralymphatic, intracavital, vaginal, transurethral, intradermal, aural, intramammary, buccal, orthotopic, intratracheal, intralesional, percutaneous, endoscopical, transmucosal, sublingual and intestinal administration.

In a particular embodiment, the compositions are administered transdermally or transmucosally. According to this embodiment, therefore, the composition is a topical composition. For instance, transdermal patches including the compositions of the present invention can be produced by techniques known in medicament delivery and applied to the skin of a mammal to be treated, after which the active ingredient(s), owing to its formulated solubility properties, migrates through the epidermis and into the dermal layers of the patient's skin, where it is taken up as part of the general circulation of the patient and finally and ultimately results in systemic distribution of the active ingredient over a desired, extended period of time. Additionally or alternatively, aerosols, creams, emulsions, foams, gels/jellies, lotions, ointments, pastes, soaps, solutions, sprays, suspensions, or tinctures, and the like including the compositions of the present invention may be topically applied to the skin of a mammal to be treated, after which the active ingredient(s), owing to its formulated solubility properties, migrates through the epidermis and into the dermal layers of the patient's skin, where it is taken up as part of the general circulation of the patient and finally and ultimately results in systemic distribution of the active ingredient over a desired, extended period of time.

### Abbreviations and Definitions

The following definitions and methods are provided to better define the present invention and to guide those of ordinary skill in the art in the practice of the present invention. Unless otherwise noted, terms are to be understood according to conventional usage by those of ordinary skill in the relevant art.

The terms “acetal” and “ketal,” as used herein alone or as part of another group, denote the moieties represented by the following formulae, respectively:

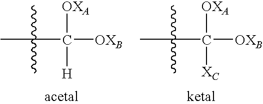

wherein X_{A} and X_{B} are independently hydrocarbyl, substituted hydrocarbyl, heterocyclo, or heteroaryl, and X_{C} is hydrocarbyl or substituted hydrocarbyl, as defined in connection with such terms, and the wavy lines represent the attachment point of the acetal or ketal moiety to another moiety or compound.

The term “acyl,” as used herein alone or as part of another group, denotes a —C(O)X_{D} radical, where X_{D} is alkyl, haloalkyl, cycloalkyl, cycloalkylalkyl, aryl, aralkyl, heteroaryl, heteroaralkyl, heterocyclyl, or heterocyclylalkyl, each as defined in connection with such term. Exemplary acyl moieties include acetyl, propionyl, benzoyl, pyridinylcarbonyl, and the like. When X_{D} of the —C(O)X_{D} radical is alkyl, for example, the radical may also be referred to as an alkylcarbonyl.

The term “acyloxy,” as used herein alone or as part of another group, denotes an acyl group as described above bonded through an oxygen linkage (—O—), e.g., —OC(O)X_{D}, wherein X_{D} is as defined in connection with the term “acyl.”

The term “alicyclic,” as used herein alone or as part of another group, denotes a non-aromatic ring, e.g., cycloalkyl or heterocyclyl ring.

The term “alkoxy,” as used herein alone or as part of another group, denotes an —OX_{E} radical, wherein X_{E} is as defined in connection with the term “alkyl.” Exemplary alkoxy moieties include methoxy, ethoxy, propoxy, or 2-propoxy, n-, iso-, or tert-butoxy, and the like.

The term “alkenoxy,” as used herein alone or as part of another group, denotes an —OX_{F} radical, wherein X_{F} is as defined in connection with the term “alkenyl.” Exemplary alkenoxy moieties include ethenoxy, propenoxy, butenoxy, hexenoxy, and the like.

The term “alkynoxy,” as used herein alone or as part of another group, denotes an —OX_{G} radical, wherein X_{G} is as defined in connection with the term “alkynyl.” Exemplary alkynoxy moieties include ethynoxy, propynoxy, butynoxy, hexynoxy, and the like.

The term “alkyl,” as used herein alone or as part of another group, denotes a linear saturated monovalent hydrocarbon radical of one to eight carbon atoms and up to 20 carbon atoms or a branched saturated monovalent hydrocarbon radical of three to eight carbon atoms. Exemplary alkyl moieties include methyl, ethyl, propyl, 2-propyl, butyl (including all isomeric forms), pentyl (including all isomeric forms), and the like.

The term “alkylene,” as used herein alone or as part of another group, denotes a linear saturated divalent hydrocarbon radical of one to eight carbon atoms or a branched saturated divalent hydrocarbon radical of three to six carbon atoms unless otherwise stated. Exemplary alkylene moieties include methylene, ethylene, propylene, 1-methylpropylene, 2-methylpropylene, butylene, pentylene, and the like.

The term “alkenyl,” as used herein alone or as part of another group, denotes groups which are preferably lower alkenyl containing from two to eight carbon atoms in the principal chain and up to 20 carbon atoms. They may be straight or branched chain or cyclic and include ethenyl, propenyl, isopropenyl, butenyl, isobutenyl, hexenyl, and the like.

The term “alkynyl,” as used herein alone or as part of another group, denotes groups which are preferably lower alkynyl containing from two to eight carbon atoms in the principal chain and up to 20 carbon atoms. They may be straight or branched chain and include ethynyl, propynyl, butynyl, isobutynyl, hexynyl, and the like.

The terms “amine” or “amino,” as used herein alone or as part of another group, represents a group of formula —N(X_{H})(X_{I}), wherein X_{H} and X_{I} are independently hydrogen, hydrocarbyl, substituted hydrocarbyl, heteroaryl, or heterocyclo, or X_{H} and X_{I} taken together form a substituted or unsubstituted alicyclic, aryl, or heterocyclic moiety, each as defined in connection with such term, typically having from 3 to 8 atoms in the ring.

The term “amido,” as used herein alone or as part of another group, represents a group of formula —CON(X_{H})(X_{I}), wherein X_{H} and X_{I} are as defined in connection with the terms “amine” or “amino.”

The term “aralkyl,” as used herein alone or as part of another group, denotes an -alkylene)-X_{J} radical, wherein X_{J} is as defined in connection with the term “aryl.”

The term “aryl,” as used herein alone or as part of another group, denotes a monovalent monocyclic or bicyclic aromatic hydrocarbon radical of 6 to 12 ring atoms, e.g., phenyl, biphenyl, naphthyl, substituted phenyl, substituted biphenyl, or substituted napthyl.

The term “cycloalkyl,” as used herein alone or as part of another group, denotes a substituted or unsubstituted, cyclic saturated monovalent bridged or non-bridged hydrocarbon radical of three to ten carbon atoms. Exemplary cycloalkyl moieties include cyclopropyl, cyclobutyl, cyclopentyl, cyclohexyl, or adamantyl.

The term “cycloalkylalkyl,” as used herein alone or as part of another group, denotes an -(alkylene)-X_{K} radical, wherein X_{K} is as defined in connection with the term “cycloalkyl” Exemplary cycloalkylalkyl moieties include cyclopropylmethyl, cyclobutylnethyl, cyclopentylethyl, or cyclohexylmethyl, and the like.

The term “cyano,” as used herein alone or as part of another group, denotes a group of formula —CN.

The term “ester,” as used herein alone or as part of another group, denotes a group of formula —COOX_{L} wherein X_{L} is alkyl or aryl, each as defined in connection with such term.

The term “ether,” as used herein alone or as part of another group, includes compounds or moieties which contain an oxygen atom bonded to two carbon atoms. For example, ether includes “alkoxyalkyl” which refers to an alkyl, alkenyl, or alkynyl group substituted with an alkoxy group.

The terms “halide,” “halo,” or “halogen,” as used herein alone or as part of another group, mean fluoro, chloro, bromo, and iodo, preferably fluoro, chloro, or bromo.

The term “haloalkyl,” as used herein alone or as part of another group, denotes an alkyl substituted with one or more halogen atoms, preferably one to five halogen atoms, preferably fluorine or chlorine, including those substituted with different halogens, e.g., —CH₂Cl, —CF₃, —CHF₂, —CF₂CF₃, —CF(CH₃)₃, and the like.

The term “heteroatom” shall mean atoms other than carbon and hydrogen.

The terms “heteroaromatic” or “heteroaryl,” as used herein alone or as part of another group, denotes an optionally substituted monovalent monocyclic or bicyclic aromatic radical of 5 to 10 ring atoms, where one or more, preferably one, two, or three, ring atoms are heteroatoms independently selected from N, O, and S, and the remaining ring atoms are carbon. Exemplary heteroaryl moieties include benzofuranyl, benzo[d]thiazolyl, isoquinolinyl, quinolinyl, thiophenyl, imidazolyl, oxazolyl, quinolinyl, furanyl, thazolyl, pyridinyl, furyl, thienyl, pyridyl, oxazolyl, pyrrolyl, indolyl, quinolinyl, isoquinolinyl, and the like. Exemplary substituents include one or more of the following groups: hydrocarbyl, substituted hydrocarbyl, keto, hydroxy, protected hydroxy, acyl, acyloxy, alkoxy, alkenoxy, alkynoxy, aryloxy, halogen, amido, amino, nitro, cyano, thiol, ketals, acetals, esters and ethers.

The term “heteroaralkyl,” as used herein alone or as part of another group, denotes an -(alkylene)-X_{M} radical, wherein X_{M} is as defined in connection with the term “heteroaryl.”

The terms “heterocyclo,” “heterocyclyl,” or “heterocyclic,” as used herein alone or as part of another group, denote a saturated or unsaturated monovalent monocyclic group of 4 to 8 ring atoms, in which one or two ring atoms are heteroatom(s), independently selected from N, O, and S(O)ₙ, where n is an integer from 0 to 2, and the remaining ring atoms are carbon atoms. Additionally, the heterocyclic ring may be fused to phenyl or heteroaryl ring, provided that the entire heterocyclic ring is not completely aromatic. Unless stated otherwise, the fused heterocyclic ring can be attached at any ring atom. More specifically, the terms “heterocyclo,” “heterocyclyl,” or “heterocyclic” include, but are not limited to, pyrrolidino, piperidino, homopiperidino, morpholino, piperazino, tetrahydropyranyl, thiomorpholino, and the like. When the heterocyclic ring has five, six or seven ring atoms, and is not fused to phenyl or heteroaryl ring, it may be referred to as a “monocyclic heterocyclyl ring.” When the heterocyclic ring is unsaturated, it can contain one or two ring double bonds, provided that the ring is not aromatic.

The term “heterocyclylalkyl,” as used herein alone or as part of another group, denotes an -(alkylene)-X_{N} radical, wherein X_{N} is as defined in connection with the term “heterocyclyl.” Exemplary heterocyclyalkyl moieties include tetrahydrofuranylmethyl, piperazinylmethyl, morpholinylethyl, and the like.

The terms “hydrocarbon” and “hydrocarbyl,” as used herein alone or as part of another group, describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms.

The term “hydroxy,” as used herein alone or as part of another group, denotes a group of formula —OH.

The term “hydroxy protecting group,” as used herein alone or as part of another group, denote a group capable of protecting a free hydroxy group (“protected hydroxy”) which, subsequent to the reaction for which protection is employed, may be removed without disturbing the remainder of the molecule. Exemplary hydroxy protecting groups include ethers (e.g., allyl, triphenylmethyl (trityl or Tr), benzyl, p-methoxybenzyl (PMB), p-methoxyphenyl (PMP)), acetals (e.g., methoxymethyl (MOM), β-methoxyethoxymethyl (MEM), tetrahydropyranyl (THP), ethoxy ethyl (EE), methylthiomethyl (MTM), 2-methoxy-2-propyl (MOP), 2-trimethylsilylethoxymethyl (SEM)), esters (e.g., benzoate (Bz), allyl carbonate, 2,2,2-trichloroethyl carbonate (Troc), 2-trimethylsilylethyl carbonate), silyl ethers (e.g., trimethylsilyl (TMS), triethylsilyl (TES), triisopropylsilyl (TIPS), triphenylsilyl (TPS), t-butyldimethylsilyl (TBDMS), t-butyldiphenylsilyl (TBDPS) and the like. A variety of protecting groups for the hydroxy group and the synthesis thereof may be found in “Protective Groups in Organic Synthesis” by T. W. Greene and P. G. M. Wuts, John Wiley & Sons, 1999.

The term “keto,” as used herein alone or as part of another group, denotes a double bonded oxygen moiety (i.e., ═O).

The term “nitro,” as used herein alone or as part of another group, denotes a group of formula —NO₂.

The “substituted hydrocarbyl” moieties, as used herein alone or as part of another group, are hydrocarbyl moieties which are substituted with at least one atom other than carbon, including moieties in which a carbon chain atom is substituted with a hetero atom such as nitrogen, oxygen, silicon, phosphorous, boron, sulfur, or a halogen atom. These substituents include halogen, heterocyclo, alkoxy, alkenoxy, alkynoxy, aryloxy, hydroxy, protected hydroxy, keto, acyl, acyloxy, nitro, amino, amido, cyano, thiol, ketals, acetals, esters, ethers, and thioethers.

The term “thioether,” as used herein alone or as part of another group, denotes compounds and moieties that contain a sulfur atom bonded to two different carbon or hetero atoms. Examples of thioethers include, but are not limited to, alkthioalkyls, alkthioalkenyls, and alkthioalkynyls. The term “alkthioalkyls” includes compounds with an alkyl, alkenyl, or alkynyl group bonded to a sulfur atom that is bonded to an alkyl group. Similarly, the term “alkthioalkenyls” and alkthioalkynyls” refer to compounds or moieties where an alkyl, alkenyl, or alkynyl group is bonded to a sulfur atom that is covalently bonded to an alkynyl group.

The term “thio,” as used herein alone or as part of another group, denotes a group of formula ═S.

The term “thiol,” as used herein alone or as part of another group, denotes a group of formula —SH.

As various changes could be made in the above compositions and methods without departing from the scope of the invention, it is intended that all matter contained in the above description shall be interpreted as illustrative and not in a limiting sense.

Having described the invention in detail, it will be apparent that modifications and variations are possible without departing the scope of the invention defined in the appended claims. Furthermore, it should be appreciated that all examples in the present disclosure are provided as non-limiting examples.

### EXAMPLE 1

### Acyclovir/1-methylimidazole

In this example, ˜1 g of Acyclovir was weighed into a 20 ml Erlenmeyer flask and 10 ml of 1-methylimidazole was added. The mixture was stirred at the appropriate temperature (0° C., 8° C., 20° C., 35° C. or 50° C.) for 30 minutes. A warm water bath or ice bath monitored with a thermocouple was used to reach the desired temperatures. Approximately 1 ml of the suspension was filtered using a syringe, filter, and vial that had been stored either in a 65° C. oven (e.g., for the 50° C. and 35° C. experiments) or in the freezer (e.g., for the 8° C. 0° C. experiments). The 20° C. experiment was filtered using a syringe, filter, and vial stored at room temperature. The filtered sample was prepared by transferring 100 μl to a 50 ml volumetric flask and diluting to volume with 0.01 N NaOH. The samples were then analyzed by HPLC, which yielded the following results:

| Sample | Sample |
|---|---|
| Temperature | mg/mL |
| 50° C. | 56.6 |
| 35° C. | 55.7 |
| 20° C. | 42.1 |
| 8° C. | 41.5 |
| 0° C. | 41.7 |

Conditions for the HPLC were as follows:
Column: Cadenza CD-18 (5 μm particle size, 4.6×150 mm, Imtakt Corp., Kyoto, Japan).
Injection Volume: 5 μl of ˜1 mg/ml intermediate in appropriate solvent.
Detection: 250 nm.
Column Temperature: 30° C.
Flow Rate: 1.0 ml/min. isocratic.
Mobile Phase: 3800 ml water; 4 ml glacial acetic acid pH-adjusted with concentrated NH₄OH; 200 ml methanol.
Run Time: 4 min.

### EXAMPLE 2

### Acyclovir/1-methylimidazole

In this example, ˜0.6 g of Acyclovir was weighed into separate 20 ml scintillation vials. The appropriate amounts of 1-methylimidazole and water were added to each vial. The vials were fitted into a holder attached to a vortex mixer and shaken for 1 hour. A small amount of each solution was then filtered with a glass fiber filter and diluted to 100 μl into a 50 ml volumetric flask with 0.01 N NaOH. The samples were then analyzed by HPLC using the same conditions as described above in example 1, which yielded the following results:

| Water | 1-methylimidazole | Sample |
|---|---|---|
| (% by volume) | (% by volume) | mg/ml |
| 100 | 0 | 1.3 |
| 95 | 5 | 6.6 |
| 90 | 10 | 4.1 |
| 85 | 15 | 8.9 |
| 75 | 25 | 10.9 |
| 50 | 50 | 13.7 |
| 25 | 75 | 11.4 |
| 10 | 90 | 27.4 |
| 0 | 100 | 57.4 |

Conditions for HPLC where the same as those described in Example 1.

### EXAMPLE 3

### Acyclovir/Imidazole

In this example, 0.5 g of imidazole was weighed into a 20 ml vial and 9.5 ml of water was added. Approximately 0.2 g of Acyclovir was added, and the vial was capped and shaken for 1 hour. A small amount of the suspension was then filtered using a glass fiber filter. Then, about 100 μL of the filtered solution was diluted to 10 ml in a volumetric flask with 0.01 N NaOH. This sample was then analyzed by HPLC using the same conditions as described in example 1, which yielded a result of 3.8 mg/ml. This procedure was repeated using approximately the same amounts as above of 4-methylimidazole. This experiment yielded a result of 5.9 mg/ml.

### EXAMPLE 4

### Acyclovir/Imidazole 2-hydroxybenzoate

In this example, approximately 1.0 g of imidazole 2-hydroxybenzoate was weighed into a 20 ml vial and 9.0 ml of water was added. The native pH was 5.29. Approximately 0.15 g of Acyclovir was added, and the vial was capped and shaken for 1 hour. A small amount of the solution was then filtered using a glass fiber filter. Then, about 100 μl of the filtered solution was diluted to 10 ml in a volumetric flask with 0.01 N NaOH in a volumetric flask. The sample was analyzed by HPLC using the same conditions as described in example 1, which yielded a result of 6.5 mg/mL. This procedure was repeated using approximately the same amounts. In this experiment, however, the pH was adjusted to 8.01 using ammonium hydroxide. This experiment yielded a result of 8.1 mg/ml.

### EXAMPLE 5

### Acyclovir/Imidazole/Salicyclic Acid

In this example, approximately 2.5 mmol each of imidazole and salicylic acid were weighed into a 20 ml vial and 9.5 ml of water was added. Native pH was 4.90. Approximately 0.12 g of Acyclovir was added and the vial was capped and shaken for 1 hour. A small amount of the solution was then filtered using a glass fiber filter. Then, about 100 μl of the filtered solution was diluted to 10 ml using 0.01 N NaOH in a volumetric flask. This sample was then analyzed by HPLC using the same conditions as described in example 1, which yielded a result of 3.4 mg/ml. This experiment was repeated using approximately the same amounts. In this experiment, however, the pH was adjusted to 9.68 using ammonium hydroxide. This experiment yielded a result of 7.2 mg/ml.

### EXAMPLE 6

### Acyclovir/Imidazole/Ibuprofen

Ibuprofen (0.8148 g, 3.95 mmol) was weighed out and placed into a 20 mL vial. Imidazole (0.2710 g, 5.03 mmol) was weighed out and also placed into the same 20 mL vial. Deionized water (9 mL) was added to the vial. The sample was vortexed for 10 min prior to the addition of acyclovir (0.2287 g). The sample was vortexed for at least 20 min prior to sampling for HPLC analysis. A small amount of the suspension was filtered using a glass fiber filter. The pH of the remaining suspension was measured at 7.01. 100 μL of the filtered solution was diluted to 10 mL in a volumetric flask with 0.01N NaOH and analyzed. The solubility of acyclovir was 2.7 mg/mL. The original suspension was pH adjusted to 7.60 using 5N NaOH solution. Sampling for HPLC was carried out as before and the solubility of acyclovir was determined to be 3.5 mg/mL.

### EXAMPLE 7

### Acyclovir/Imidazole/Naproxen

Ibuprofen (0.7399 g, 3.21 mmol) was weighed out and placed into a 20 mL vial. Imidazole (0.2277 g, 3.34 mmol) was weighed out and also placed into the same 20 mL vial. Deionized water (9 mL) was added to the vial. The sample was vortexed for 10 min prior to the addition of acyclovir (0.2183 g). The sample was vortexed for at least 20 min prior to sampling for HPLC analysis. A small amount of the suspension was filtered using a glass fiber filter. The pH of the remaining suspension was measured at 7.37. 100 μL of the filtered solution was diluted to 10 mL in a volumetric flask with 0.01N NaOH and analyzed. The solubility of acyclovir was 4.9 mg/mL. The original suspension was pH adjusted to 7.70 using 5N NaOH solution. Sampling for HPLC was carried out as before and the solubility of acyclovir was determined to be 10.6 mg/mL.

### EXAMPLE 8

### Acyclovir/Salicylic Acid Imidazolium Salt

Salicyclic acid imidazolium salt (1.0375 g, 5.03 mmol) was weighed out and placed into a 20 mL vial. Deionized water (9 mL) was added to the vial. The sample was vortexed for 10 min prior to the addition of acyclovir (0.2390 g). The sample was vortexed for at least 20 min prior to sampling for HPLC analysis. A small amount of the suspension was filtered using a glass fiber filter. The pH of the remaining suspension was measured at 5.28. 100 μL of the filtered solution was diluted to 10 mL in a volumetric flask with 0.01N NaOH and analyzed. The solubility of acyclovir was 7.1 mg/mL.

### EXAMPLE 9

### Acyclovir/Imidazole/Ketoprofen

Ketoprofen (0.6028 g, 2.37 mmol) was weighed out and placed into a 20 mL vial. Imidazole (0.1638 g, 2.41 mmol) was weighed out and also placed into the 20 mL vial. Deionized water (9 mL) was added to the vial. The sample was vortexed for 10 min prior to the addition of acyclovir (0.2340 g). The sample was vortexed for at least 20 min prior to sampling for HPLC analysis. A small amount of the suspension was filtered using a glass fiber filter. The pH of the remaining suspension was measured at 5.60. 100 μL of the filtered solution was diluted to 10 mL in a volumetric flask with 0.01N NaOH and analyzed. The solubility of acyclovir was 3.9 mg/mL. The original suspension was pH adjusted to 8.06 using 5N NaOH solution. Sampling for HPLC was carried out as before and the solubility of acyclovir was determined to be 5.0 mg/mL.

### EXAMPLE 10

### Acyclovir/Imidazole/Flurbiprofen

Flurbiprofen (0.5123 g, 2.10 mmol) was weighed out and placed into a 20 mL vial. Imidazole (0.1461 g, 2.14 mmol) was weighed out and also placed into the same 20 mL vial. Deionized water (9 mL) was added to the vial. The sample was vortexed for 10 min prior to the addition of acyclovir (0.2034 g). The sample was vortexed for at least 20 min prior to sampling for HPLC analysis. A small amount of the suspension was filtered using a glass fiber filter. The pH of the remaining suspension was measured at 7.02. 100 μL of the filtered solution was diluted to 10 mL in a volumetric flask with 0.01N NaOH and analyzed. The solubility of acyclovir was 3.3 mg/mL. The original suspension was pH adjusted to 7.72 using 5N NaOH solution. Sampling for HPLC was carried out as before and the solubility of acyclovir was determined to be 5.1 mg/mL.

### EXAMPLE 11

### Acyclovir/Imidazole/Sulindac

Sulindac (0.5314 g, 1.49 mmol) was weighed out and placed into a 20 mL vial. Imidazole (0.1063 g, 1.56 mmol) was weighed out and also placed into the same 20 mL vial. Deionized water (9 mL) was added to the vial. The sample was vortexed for 10 min prior to the addition of acyclovir (0.2226 g). The sample was vortexed for at least 20 min prior to sampling for HPLC analysis. A small amount of the suspension was filtered using a glass fiber filter. The pH of the remaining suspension was measured at 6.09. 10 μL of the filtered solution was diluted to 10 mL in a volumetric flask with 0.01N NaOH and analyzed. The solubility of acyclovir was 4.1 mg/mL. The original suspension was pH adjusted to 7.84 using 5N NaOH solution. Sampling for HPLC was carried out as before and the solubility of acyclovir was determined to be 5.1 mg/mL.

### EXAMPLE 12

### Acyclovir/Sodium Imidazole/Naproxen

Naproxen (0.5963 g, 2.59 mmol) was weighed out and placed into a 20 mL vial. Sodium imidazole (0.227 g, 2.52 mmol) was weighed out and also placed into the same 20 mL vial. Deionized water (9 mL) was added to the vial. The sample was vortexed for 10 min prior to the addition of acyclovir (0.2083 g). The sample was vortexed for at least 20 min prior to sampling for HPLC analysis. A small amount of the suspension was filtered using a glass fiber filter. The pH of the remaining suspension was measured at 7.72. 100 μL of the filtered solution was diluted to 10 mL in a volumetric flask with 0.01N NaOH and analyzed. The solubility of acyclovir was 10.6 mg/mL.

### EXAMPLE 13

### Acyclovir/1-N-methylimidazole/Sulindac

Sulindac (0.4578 g, 1.28 mmol) was weighed out and placed into a 20 mL vial. 1-N-methylImidazole (0.1068 g, 1.30 mmol) was weighed out and also placed into the same 20 mL vial. Deionized water (9 mL) was added to the vial. The sample was vortexed for 10 min prior to the addition of acyclovir (0.2071 g). The sample was vortexed for at least 20 min prior to sampling for HPLC analysis. A small amount of the suspension was filtered using a glass fiber filter. The pH of the remaining suspension was measured at 7.08. 100 μL of the filtered solution was diluted to 10 mL in a volumetric flask with 0.01N NaOH and analyzed. The solubility of acyclovir was 3.9 mg/mL.

### EXAMPLE 14

### Acyclovir/Diclofenac Sodium Salt

Diclofenac sodium salt was weighed out and placed into a 20 mL vial. Deionized water (9 mL) was added to the vial. The sample was vortexed for 10 min prior to the addition of acyclovir. The suspension was vortexed for at least 20 min prior to sampling for HPLC analysis. A small amount of the suspension was filtered using a glass fiber filter. 100 μL of the filtered solution was diluted to 10 mL in a volumetric flask with 0.01N NaOH and analyzed. The solubility of acyclovir was 3.5 mg/mL at a concentration of Diclofenac Sodium salt of 21.9 mg/mL.

### EXAMPLE 15

### Acyclovir/Imidazole/Diclofenac Sodium Salt

Diclofenac sodium salt was weighed out and placed into a 20 mL vial. Deionized water (9 mL) was added to the vial. Imidazole was weighed out and also placed into the same 20 mL vial. The sample was vortexed for 10 min prior to the addition of acyclovir. The suspension was vortexed for at least 20 min prior to sampling for HPLC analysis. A small amount of the suspension was filtered using a glass fiber filter. 100 μL of the filtered solution was diluted to 10 mL in a volumetric flask with 0.01N NaOH and analyzed. The solubility of acyclovir was 5.6 mg/mL at a concentration of Diclofenac sodium salt of 26.8 mg/mL and imidazole concentration of 22.6 mg/mL.

### EXAMPLE 16

### 3′-Azido-3′-Deoxythymidine (AZT)

### (A) AZT Solubility in Water

AZT (0.1027 g) was weighed out and placed into a 10 mL vial. Deionized water (1 mL) was added to the vial. The sample was vortexed for at least 20 min prior to sampling for UPLC analysis. A small amount of the suspension was filtered using a glass fiber filter. 100 μL of the filtered solution was diluted to 10 mL in a volumetric flask with 0.01N NaOH and analyzed. The solubility of AZT was 16.8 mg/mL.

### (B) Imidazole and AZT Solubility in Water

AZT (0.1027 g) was weighed out and placed into a 10 mL vial. Imidazole (0.0180 g) was weighed out and placed into the 10 mL vial. Deionized water (1 mL) was added to the vial. The sample was vortexed for at least 20 min prior to sampling for analysis. A small amount of the suspension was filtered using a glass fiber filter. 100 μL of the filtered solution was diluted to 50 mL in a volumetric flask with 0.01N NaOH and analyzed by UPLC. The solubility of AZT was 23.1 mg/mL.

A sample of AZT (0.1044 g) using the same sample preparation with more imidazole (0.0363 g) analyzed at 29.2 mg/mL concentration in water.

### (C) 1-N-methylimidazole and AZT Solubility in Water

AZT (0.1027 g) was weighed out and placed into a 10 mL vial. 1-N-methylimidazole (0.0426 g) was weighed out and placed into the 10 mL vial. Deionized water (1 mL) was added to the vial. The sample was vortexed for at least 20 min prior to sampling for analysis. A small amount of the suspension was filtered using a glass fiber filter. 100μL of the filtered solution was diluted to 50 mL in a volumetric flask with 0.01N NaOH and analyzed by UPLC. The solubility of AZT was 30.1 mg/mL.

(D) Salicyclic acid Imidazolium Salt and AZT Solubility in Water

AZT (0.0606 g) was weighed out and placed into a 10 mL vial. Salicyclic acid Imidazolium salt (0.0405 g) was weighed out and placed into the 10 mL vial. Deionized water (1 mL) was added to the vial. The sample was vortexed for at least 20 min prior to sampling for analysis. A small amount of the suspension was filtered using a glass fiber filter. 100 μL of the filtered solution was diluted to 50 mL in a volumetric flask with 0.01N NaOH and analyzed by UPLC. The solubility of AZT was 36.0 mg/mL.

### (E) Naproxen, Imidazole and AZT Solubility in Water

AZT (0.0633 g) was weighed out and placed into a 10 mL vial. Naproxen (0.0306 g) was weighed out and placed into the 10 mL vial. Deionized water (1 mL) was added to the vial. A thick suspension resulted at room temperature. The sample was vortexed for at least 20 min prior to sampling for analysis. A small amount of the suspension was filtered using a glass fiber filter. 100 μL of the filtered solution was diluted to 50 mL in a volumetric flask with 0.01N NaOH and analyzed by UPLC. The solubility of AZT was 17.7 mg/mL.

Imidazole (0.0122 g) was weighed out and placed into the 10 mL vial containing the suspension. The sample was vortexed for at least 20 min prior to sampling for analysis. A solution resulted. A small amount of the solution was filtered using a glass fiber filter. 100 μL of the filtered solution was diluted to 50 mL in a volumetric flask with 0.O1N NaOH and analyzed by UPLC. The solubility of AZT increased to 54.0 mg/ML.

### (F) Sodium Benzoate, Imidazole and AZT Solubility in Water

AZT (0.0502 g) was weighed out and placed into a 10 mL vial. Sodium benzoate (0.0541 g) was weighed out and placed into the 10 mL vial. Deionized water (1 mL) was added to the vial. A suspension resulted at room temperature. The sample was vortexed for at least 20 min prior to sampling for analysis. A small amount of the suspension was filtered using a glass fiber filter. 100 μL of the filtered solution was diluted to 50 mL in a volumetric flask with 0.01N NaOH and analyzed by UPLC. The solubility of AZT was 28.8 mg/mL.

Imidazole (0.0144 g) was weighed out and placed into the 10 mL vial containing the suspension. The sample was vortexed for at least 20 min prior to sampling for analysis. A solution resulted. A small amount of the solution was filtered using a glass fiber filter. 100 μL of the filtered solution was diluted to 50 mL in a volumetric flask with 0.01N NaOH and analyzed by UPLC. The solubility of AZT increased to 48.4 mg/mL.

### (G) Naproxen, Imidazole and AZT Solubility in Water

AZT (0.0633 g) was weighed out and placed into a 10 mL vial. Naproxen (0.0306 g) was weighed out and placed into the 10 mL vial. Deionized water (1 mL) was added to the vial. A thick suspension resulted at room temperature. The sample was vortexed for at least 20 min prior to sampling for analysis. A small amount of the suspension was filtered using a glass fiber filter. 100 μL of the filtered solution was diluted to 50 mL in a volumetric flask with 0.01N NaOH and analyzed by UPLC. The solubility of AZT was 17.7 mg/mL.

Imidazole (0.0122 g) was weighed out and placed into the 10 mL vial containing the suspension. The sample was vortexed for at least 20 min prior to sampling for analysis. A solution resulted. A small amount of the solution was filtered using a glass fiber filter. 100 μL of the filtered solution was diluted to 50 mL in a volumetric flask with 0.01N NaOH and analyzed by UPLC. The solubility of AZT increased to 54.0 mg/mL.

(H) Naproxen, Imidazole and AZT Solubility in n-Octanol

AZT (0.0549 g) was weighed out and placed into a 10 mL vial. n-Octanol (1 mL) was added into the vial. The sample was vortexed at room temperature for at least 20 min prior to sampling for analysis. A small amount of the suspension was filtered using a glass fiber filter. 100 μL of the filtered solution was diluted to 50 mL in a volumetric flask with 0.01N NaOH and analyzed by UPLC. The solubility of AZT was 6.7 mg/mL.

AZT (0.0513 g) was weighed out and placed into a 10 mL vial. Imidazole (0.0171 g) was weighed out and placed into the 10 mL vial. n-Octanol (1 mL) was added into the vial. The sample was vortexed for at least 20 min prior to sampling for analysis. A small amount of the solution was filtered using a glass fiber filter. 100 μL of the filtered solution was diluted to 50 mL in a volumetric flask with 0.01N NaOH and analyzed by UPLC. The solubility of AZT was 6.9 mg/mL.

AZT (0.0470 g) was weighed out and placed into a 10 mL vial. Imidazole (0.0143 g) was weighed out and placed into the 10 mL vial. Naproxen (0.0330 g) was weighed out and also placed into the 10 mL vial. n-Octanol (1 mL) was added into the vial. The sample was vortexed for at least 20 min prior to sampling for analysis. A small amount of the solution was filtered using a glass fiber filter. 100 μL of the filtered solution was diluted to 50 mL in a volumetric flask with 0.01N NaOH and analyzed by UPLC. The solubility of AZT increased to 15.7 mg/mL.

### (I) Naproxen, Imidazole and 5-Fluorouracil Solubility in Water

5-Fluorouracil (0.0809 g) was weighed out and placed into a 10 mL vial. Deionized water (1 mL) was added to the vial. The sample was vortexed for at least 20 min at room temperature prior to sampling for analysis. A small amount of the suspension was filtered using a glass fiber filter. 100 μL of the filtered solution was diluted to 50 mL in a volumetric flask with 0.01N NaOH and analyzed by UPLC. The solubility of 5-fluorouracil was 10.7 mg/mL.

5-Fluorouracil (0.0887 g) was weighed out and placed into a 10 mL vial. 1-N-methylimidazole (0.1 mL) was transferred by pipette into the 10 mL vial. Deionized water (1 mL) was added to the vial. The sample was vortexed for at least 20 min prior to sampling for analysis. A small amount of the solution was filtered using a glass fiber filter. 100 μL of the filtered solution was diluted to 50 mL in a volumetric flask with 0.01N NaOH and analyzed by UPLC. The solubility of 5-fluorouracil increased to 31.4 mg/mL.

Naproxen (0.0535 g) was weighed out and placed into the 10 mL vial. 5-Fluorouracil (0.0836 g) was weighed out and placed into a 10 mL vial. 1-N-methylimidazole (0.1 mL) was transferred by pipette into the 10 mL vial. Deionized water (1 mL) was added to the vial. The sample was vortexed for at least 20 min prior to sampling for analysis. A small amount of the solution was filtered using a glass fiber filter. 100 μL of the filtered solution was diluted to 50 mL in a volumetric flask with 0.01N NaOH and analyzed by UPLC. The solubility of 5-fluorouracil was 32.4 mg/mL.

### (J) Naproxen, Imidazole and Ganciclovir Solubility in Water

Ganciclovir (0.0086 g) was weighed out and placed into a 10 mL vial. Deionized water (0.5 mL) was added to the vial. The sample was vortexed for at least 20 min at room temperature prior to sampling for analysis. A small amount of the suspension was filtered using a glass fiber filter. 100 μL of the filtered solution was diluted to 50 mL in a volumetric flask with 0.01N NaOH and analyzed by UPLC. The solubility of ganciclovir was 2.1 mg/mL.

Ganciclovir (0.0084 g) was weighed out and placed into a 10 mL vial. Imidazole (0.0053 g) was weighed out and placed into the 10 mL vial. Deionized water (0.5 mL) was added to the vial. The sample was vortexed for at least 20 min prior to sampling for analysis. A small amount of the solution was filtered using a glass fiber filter. 100 μL of the filtered solution was diluted to 50 mL in a volumetric flask with 0.01N NaOH and analyzed by UPLC. The solubility of ganciclovir increased to 3.0 mg/mL.

Naproxen (0.0071 g) was weighed out and placed into the 10 mL vial. Ganciclovir (0.0100 g) was weighed out and placed into a 10 mL Imidazole (0.0044 g) was weighed out and placed into the 10 mL vial. Deionized water (0.5 mL) was added to the vial. The sample was vortexed for at least 20 min prior to sampling for analysis. A small amount of the solution was filtered using a glass fiber filter. 100 μL of the filtered solution was diluted to 50 mL in a volumetric flask with 0.01N NaOH and analyzed by UPLC. The solubility of ganciclovir increased to 5.2 mg/mL.

### (K) 2-Napthalenesulfonic Acid Sodium Salt Imidazole and AZT Solubility in Water

AZT (0.0567 g) was weighed out and placed into a 10 mL vial. 2-Napthalenesulfonic acid sodium salt (0.0313 g) was also weighed out and placed in the 10 mL vial. Deionized water (1 mL) was added to the vial. The sample was vortexed for at least 20 min at room temperature prior to sampling for analysis. A small amount of the suspension was filtered using a glass fiber filter. 100 μL of the filtered solution was diluted to 50 mL in a volumetric flask with 0.01N NaOH and analyzed by UPLC. The solubility of AZT was 40.1 mg/mL.

AZT (0.0623 g) was weighed out and placed into a 10 mL vial. 2-Napthalenesulfonic acid sodium salt (0.0224 g) was weighed out and placed in the 10 mL vial. Imidazole (0.0165 g) was weighed out and also placed into the 10 mL vial. Deionized water (1 mL) was added to the vial. The sample was vortexed for at least 20 min at room temperature prior to sampling for analysis. A small amount of the solution was filtered using a glass fiber filter. 100 μL of the filtered solution was diluted to 50 mL in a volumetric flask with 0.01N NaOH and analyzed by UPLC. The solubility of AZT increased to 56.7 mg/mL.

In view of the above, it will be seen that the several objects of the invention are achieved and other advantageous results attained.

What is claimed is:

1. A composition comprising: (A) an antiviral compound or a pharmaceutically acceptable salt thereof; and (B) an agent selected from the group consisting of a substituted or unsubstituted imidazole or a pharmaceutically acceptable salt thereof; a non-steroidal anti inflammatory agent or a pharmaceutically acceptable salt thereof; an amino acid or a pharmaceutically acceptable salt thereof; a carboxylic acid or a pharmaceutically acceptable salt thereof; a sulfonic acid or a pharmaceutically acceptable salt thereof; and a combination thereof; wherein:
the antiviral compound corresponds to Formulae (1) or (2): 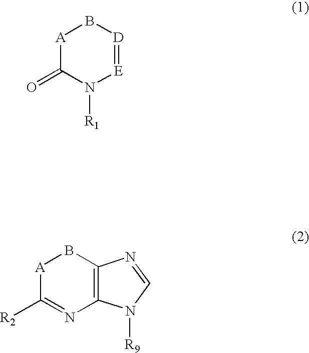
the substituted or unsubstituted imidazole corresponds to Formula (3): 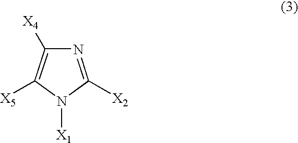
-A-B— correspond to: 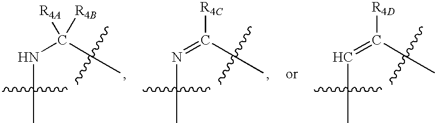
-D=E- correspond to: 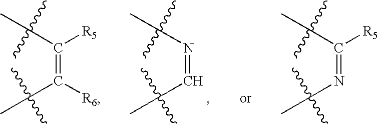
R₁ is hydrogen, heterocyclo, hydrocarbyl, or substituted hydrocarbyl;
R₂ is hydrogen, hydroxy, amino, or halo;
R_{4A} and R_{4B} are hydrogen or together form keto;
R_{4C} is hydrogen, hydroxy, amino, or alkoxy;
R_{4D} is hydroxy or alkoxy;
R₅ is hydrogen, hydrocarbyl, substituted hydrocarbyl, alkoxy, or halo;
R₆ is hydrogen or amino;
R₉ is hydrocarbyl, substituted hydrocarbyl, or heterocyclo;
X₁ is hydrogen, hydrocarbyl, substituted hydrocarbyl, or acyl; and
X₂, X₄, and X₅ are independently hydrogen, hydrocarbyl, substituted hydrocarbyl, heterocyclo, halo, alkoxy, amino, or nitro;
provided, however, that when the antiviral compound corresponds to Formula (2) wherein R₉ is —CH₂OCH₂CH₂OH, then at least one of X₁, X₂, and X₅ are other than hydrogen, or 4 is other than —CH₂—NH₂.

2. A composition comprising: (A) an antiviral compound or a pharmaceutically acceptable salt thereof; and (B) a substituted or unsubstituted imidazole or a pharmaceutically acceptable salt thereof; wherein:
the antiviral compound corresponds to Formulae (t) or (2): 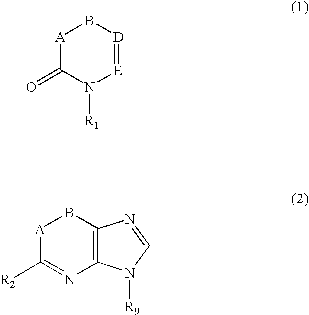
the substituted or unsubstituted imidazole corresponds to Formula (3): 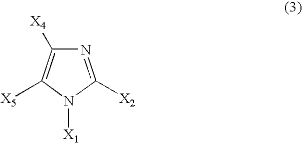
-A-B— correspond to: 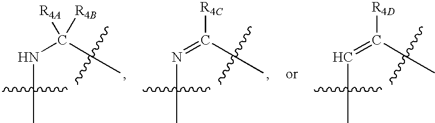
-D=E- correspond to: 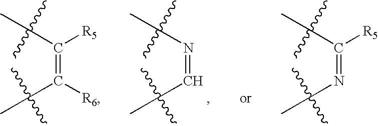
R₁ is hydrogen, heterocyclo, hydrocarbyl, or substituted hydrocarbyl;
R₂ is hydrogen, hydroxy, amino, or halo;
R_{4A} and R_{4B} are hydrogen or together form keto;
R_{4C} is hydrogen, hydroxy, amino, or alkoxy;
R_{4D} is hydroxy or alkoxy;
R₅ is hydrogen, hydrocarbyl, substituted hydrocarbyl, alkoxy, or halo;
R₆ is hydrogen or amino;
R₉ is hydrocarbyl, substituted hydrocarbyl, or heterocyclo;
X₁ is hydrogen, hydrocarbyl, substituted hydrocarbyl, or acyl; and
X₂, X₄, and X₅ are independently hydrogen, hydrocarbyl, substituted hydrocarbyl, heterocyclo, halo, alkoxy, amino, or nitro;
provided, however, that when the antiviral compound is a purine derivative corresponding to Formula (2) and R₉ is —CH₂OCH₂CH₂OH, then at least one of X₁, X₂, and X₅ are other than hydrogen, or X₄ is other than —CH₂—NH₂.

3. A composition comprising: (A) an antiviral compound or a pharmaceutically acceptable salt thereof; (B) a substituted or unsubstituted imidazole or a pharmaceutically acceptable salt thereof; and (C) a non-steroidal anti-inflammatory agent or a pharmaceutically acceptable salt thereof; wherein:
the antiviral compound corresponds to Formulae (1) or (2): 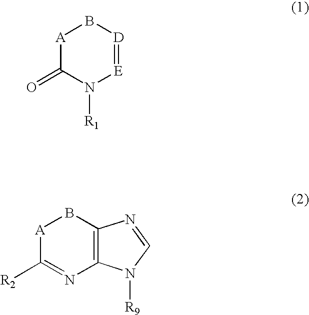
the substituted or unsubstituted imidazole corresponds to Formula (3): 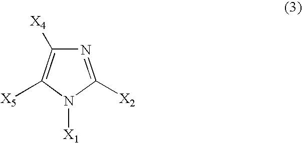
-A-B— correspond to: 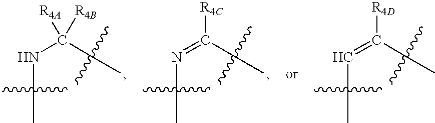
-D=E- correspond to: 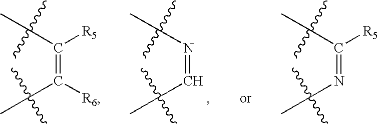
R₁ is hydrogen, heterocyclo, hydrocarbyl, or substituted hydrocarbyl;
R₂ is hydrogen, hydroxy, amino, or halo;
R_{4A} and R_{4B} are hydrogen or together form keto;
R_{4C} is hydrogen, hydroxy, amino, or alkoxy;
R_{4D} is hydroxy or alkoxy;
R₅ is hydrogen, hydrocarbyl, substituted hydrocarbyl, alkoxy, or halo;
R₆ is hydrogen or amino;
R₉ is hydrocarbyl, substituted hydrocarbyl, or heterocyclo;
X₁ is hydrogen, hydrocarbyl, substituted hydrocarbyl, or acyl; and
X₂, X₄, and X₅ are independently hydrogen, hydrocarbyl, substituted hydrocarbyl, heterocyclo, halo, alkoxy, amino, or nitro;
provided, however, that when the antiviral compound is a purine derivative corresponding to Formula (2) and R₉ is —CH₂OCH₂CH₂OH, then at least one of X₁, X₂, and X5 are other than hydrogen, or X₄ is other than —CH₂—NH₂.

4. A composition comprising: (A) an antiviral compound or a pharmaceutically acceptable salt thereof; and (B) a non-steroidal anti-inflammatory agent or a pharmaceutically acceptable salt thereof; wherein:
the antiviral compound corresponds to Formulae (1) or (2): 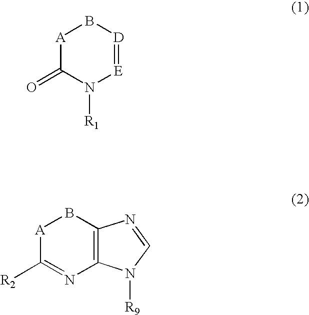
-A-B— correspond to: 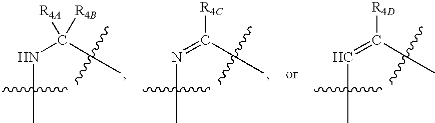
-D=E- correspond to: 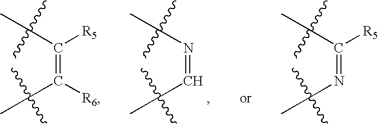
R₁ is hydrogen, heterocyclo, hydrocarbyl, or substituted hydrocarbyl;
R₂ is hydrogen, hydroxy, amino, or halo;
R_{4A} and R_{4B} are hydrogen or together form keto;
R_{4C} is hydrogen, hydroxy, amino, or alkoxy;
R_{4D} is hydroxy or alkoxy;
R₅ is hydrogen, hydrocarbyl, substituted hydrocarbyl, alkoxy, or halo;
R₆ is hydrogen or amino; and
R₉ is hydrocarbyl, substituted hydrocarbyl, or heterocyclo.

5. A composition comprising: (A) an antiviral compound or a pharmaceutically acceptable salt thereof; and (B) an agent selected from the group consisting of a carboxylic acid or a pharmaceutically acceptable salt thereof; a sulfonic acid or a pharmaceutically acceptable salt thereof; and a combination thereof; wherein:
the antiviral compound corresponds to Formulae (1) or (2): 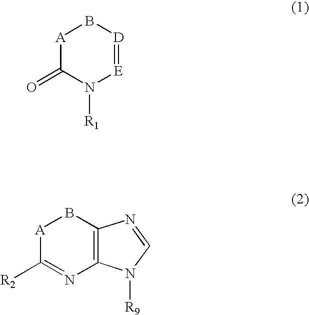
-A-B— correspond to: 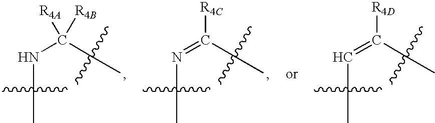
-D=E- correspond to: 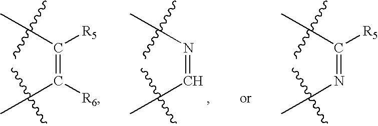
R₁ is hydrogen, heterocyclo, hydrocarbyl, or substituted hydrocarbyl;
R₂ is hydrogen, hydroxy, amino, or halo;
R_{4A} and R_{4B} are hydrogen or together form keto;
R_{4C} is hydrogen, hydroxy, amino, or alkoxy;
R_{4D} is hydroxy or alkoxy;
R₅ is hydrogen, hydrocarbyl, substituted hydrocarbyl, alkoxy, or halo;
R₆ is hydrogen or amino; and
R₉ is hydrocarbyl, substituted hydrocarbyl, or heterocyclo.

6. A method for the prophylaxis or treatment of a viral infection, the method comprising administering to a mammal in need of such prophylaxis or treatment a prophylactic or therapeutic amount of a composition comprising: (A) an antiviral compound or a pharmaceutically acceptable salt thereof; and (B) an agent selected from the group consisting of a substituted or unsubstituted imidazole or a pharmaceutically acceptable salt thereof; a non-steroidal anti-inflammatory agent or a pharmaceutically acceptable salt thereof; an amino acid or a pharmaceutically acceptable salt thereof; a carboxylic acid or a pharmaceutically acceptable salt thereof; a sulfonic acid or a pharmaceutically acceptable salt thereof; and a combination thereof; wherein:
the antiviral compound corresponds to Formulae (1) or (2): 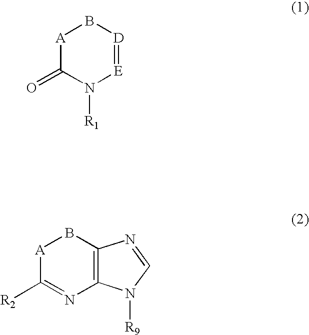
the substituted or unsubstituted imidazole corresponds to Formula (3): 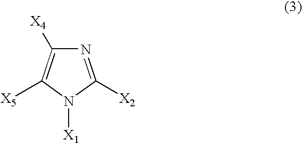
-A-B— correspond to: 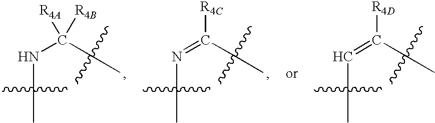
-D=E- correspond to: 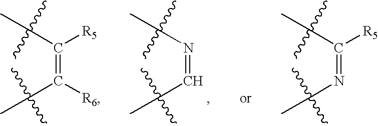
R₁ is hydrogen, heterocyclo, hydrocarbyl, or substituted hydrocarbyl;
R₂ is hydrogen, hydroxy, amino, or halo;
R_{4A} and R_{4B} are hydrogen or together form keto;
R_{4C} is hydrogen, hydroxy, amino, or alkoxy;
R_{4D} is hydroxy or alkoxy;
R₅ is hydrogen, hydrocarbyl, substituted hydrocarbyl, alkoxy, or halo;
R₆ is hydrogen or amino;
R₉ is hydrocarbyl, substituted hydrocarbyl, or heterocyclo;
X₁ is hydrogen, hydrocarbyl, substituted hydrocarbyl, or acyl; and
X₂, X₄, and X₅ are independently hydrogen, hydrocarbyl, substituted hydrocarbyl, heterocyclo, halo, alkoxy, amino, or nitro;
provided, however, that when the antiviral compound is a purine derivative corresponding to Formula (2) and R₉ is —CH₂OCH₂CH₂OH, then at least one of X₁, X₂, and X₅ are other than hydrogen, or X4 is other than —CH₂—NH₂.

7. The composition of claim 1 wherein the antiviral compound is a pyrimidine derivative corresponding to Formula (1A): 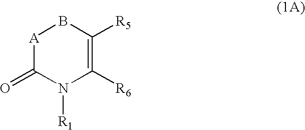
wherein:
-A-B— is: 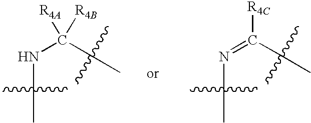
R₁ is hydrogen, heterocyclo, hydrocarbyl, or substituted hydrocarbyl;
R_{4A} and R_{4B} are hydrogen or together form keto;
R_{4C} is hydrogen, hydroxy, amino, or alkoxy;
R₅ is hydrogen, hydrocarbyl, substituted hydrocarbyl, alkoxy, or halo; and
R₆ is hydrogen or amino.

8. The composition of claim 7 wherein R₁ is: 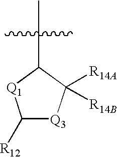
-Q₁- is —O— or —S—;
-Q₃- is —O—, —S—, or —CH(R₁₃)—
R₁₂ and R_{14A} are independently acyloxy, alkoxy, halo, hydrogen, hydroxy, hydrocarbyl, substituted hydrocarbyl, or N₃;
R₁₃ is acyl, acyloxy, halo, hydrogen, hydroxy, hydrocarbyl, substituted hydrocarbyl, or N₃, or together with R_{14B} forms a bond; and
R_{14B} is hydrogen or halo, or together with R₁₃ forms a bond.

9. The composition of claim 7 wherein R₁ is —(CH₂)ₙ—CH(R₁₁₁)(R₁₁₂), R₁₁₁ and R₁₁₂ are independently acyloxy, hydrocarbyl, substituted hydrocarbyl, or —O—(CH₂)ₙ—P(═O)(OH)₂, and each n is independently 1, 2, 3, or 4.

10. The composition of claim 9 wherein R₅ is hydrogen, substituted or unsubstituted alkyl, alkenyl, or alkynyl, or halo.

11. The composition of claim 10 wherein R₆ is hydrogen or —NH₂.

12. The composition of claim 1 wherein the antiviral compound is a purine derivative corresponding to Formula (2): 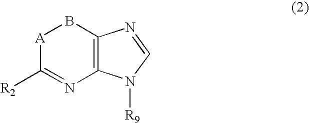
wherein:
-A-B— is: 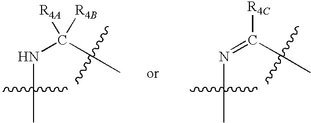
R₂ is hydrogen, amino, or halo;
R_{4A} and R_{4B} are hydrogen or together form keto;
R_{4C} is hydroxy, amino, or alkoxy; and
R₉ is hydrocarbyl, substituted hydrocarbyl, or heterocyclo.

13. The composition of claim 12 wherein:
R₉ is substituted cycloalkyl, substituted cycloalkenyl, —(CH₂)ₙ—CH(—(CH₂)ₘ—CH₃)(—O—(CH₂)ₙ—P(═O)(OH)₂), —(CH₂)ₙ—R₉₁—R₉₂, or heterocyclo;
R₉₁ is —O— or —(CH₂)ₙ—;
R₉₂ is —(CH₂)ₘ—CH(R₉₃)(R₉₄);
R₉₃ and R₉₄ are independently hydrogen, —(CH₂)ₙ—OR₉₅, or —(CH₂)ₙ—R₉₆,
R₉₅ is hydrogen or acyl;
R₉₆ is an amino acid ester;
m is 0, 1, 2, or 3; and
each n is independently 1, 2, 3, or 4.

14. The composition of claim 12 wherein R₉ is: 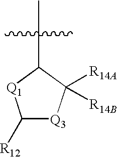
-Q₁- is —O— or —S—;
-Q₃- is —O—, —S—, or —CH(R₁₃)—
R₁₂ and R_{14A} are independently acyloxy, alkoxy, halo, hydrogen, hydroxy, hydrocarbyl, substituted hydrocarbyl, or N₃;
R₁₃ is acyl, acyloxy, halo, hydrogen, hydroxy, hydrocarbyl, substituted hydrocarbyl, or N₃, or together with R_{14B} forms a bond; and
R_{14B} is hydrogen or halo, or together with R₁₃ forms a bond.

15. The composition of claim 13 wherein R₉ is: 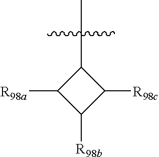
wherein R₉₈ₐ, R_{98b}, and R_{98c} are independently hydrogen, hydroxy, hydrocarbyl, or substituted hydrocarbyl.

16. The composition of claim 13 wherein R₉ is: 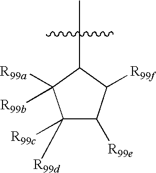
wherein R₉₉ₐ, R_{99d}, and R_{99c}, and R_{99f} are independently hydrogen, hydroxy, hydrocarbyl, or substituted hydrocarbyl, and R_{99b} and R_{99c} are each hydrogen or together form a bond.

17. The composition of claim 12 wherein -A-B— corresponds to: 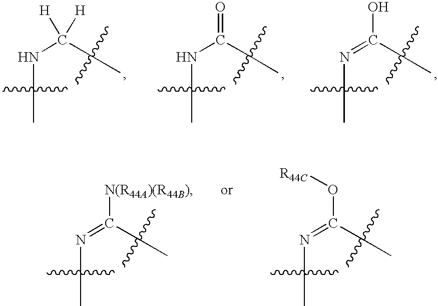
wherein R_{44A} and R_{44B} are independently hydrogen, hydrocarbyl, substituted hydrocarbyl, heteroaryl, or heterocyclo, or R_{44A} and R_{44B} taken together form a substituted or unsubstituted alicyclic, aryl, or heterocyclic moiety, and R_{44C} is alkyl.

18. The composition of claim 17 wherein R₂ is hydrogen, —NH₂, chloro, or fluoro.

19. The composition of claim 1 wherein
X₁ is hydrogen, alkyl, aryl, —C(═O)—X₆, or —(CH₂)ₙ—X₇;
X₂, R₄, and X₅ are independently hydrogen, alkyl, aryl, —C(═O)—X₆, —(CH₂)ₙ—X₇, —NH₂, or —NO₃;
each X₆ is independently hydrogen, hydroxy, methyl, ethyl, propyl, butyl, phenyl, benzyl, or —NH₂;
each X₇ is independently methoxy, ethoxy, ethenoxy, propenoxy, hydroxy, halo, or —NH₂; and
each n is independently 1, 2, 3, or 4.

20. The composition of claim 1 wherein one of X₁, X₂, X₄, and X₅ is alkyl and the others of X₁, X₂, X₄, and X₅ are each hydrogen.

21. The composition of claim 1 wherein the non-steroidal anti-inflammatory agent is selected from the group consisting of a salicylate derivative, an arylpropionic acid derivative (i.e., a profen), an arylbutanoic acid derivative (i.e., a bufen), a pyrazolidine derivative, an N-arylanthranilic acid derivative (i.e., a fenamic acid derivative), an oxicam, a sulfonanilide, an arylalkanoic acid derivative, an acetic acid derivative, an acetamide derivative, a pharmaceutically acceptable salt thereof, and a combination thereof.

22. The composition of claim 21 wherein the non-steroidal anti-inflammatory agent is a salicylate derivative selected from the group consisting of acetylsalicylic acid, salicylic acid, 5-aminosalicylic acid, sulfanilic acid, a pharmaceutically acceptable salt thereof, and a combination thereof.

23. The composition of claim 21 wherein the non-steroidal anti-inflammatory agent is an arylpropionic acid derivative or an arylbutanoic acid derivative selected from the group consisting of aminoprofen, benoxaprofen, butibufen, carprofen, fenbufen, fenoprofen, flurbiprofen, ibuprofen, indoprofen, ketoprofen, ketorolac, loxoprofen, naproxen, oxaprozin, pirprofen, pranoprofen, suprofen, tiaprofenic acid, a pharmaceutically acceptable salt thereof, and a combination thereof.

24. The composition of claim 21 wherein the non-steroidal anti-inflammatory agent is an arylalkanoic acid selected from the group consisting of diclofenac, ibufenac, aceclofenac, bromfenac, etodolac, indomethacin, nalbumetone, sulindac, tolmetin, zomepirac, a pharmaceutically acceptable salt thereof, and a combination thereof.
